(19) United States
(12) Patent Application Publication
(10) Pub. No.: US 2017/0029362 A1
(43) Pub. Date: Feb. 2, 2017

(54) LUMINESCENT COMPOUNDS

(71) Applicant: E I DU PONT DE NEMOURS AND COMPANY, Wilmington, DE (US)

(72) Inventors: MICHAEL HENRY HOWARD, JR., MONTCHANIN, DE (US); VIACHESLAV V. DIEV, WILMINGTON, DE (US); WEIYING GAO, LANDENBERG, PA (US); WEISHI WU, LANDENBERG, PA (US)

(21) Appl. No.: 15/215,303

(22) Filed: Jul. 20, 2016

Related U.S. Application Data
(60) Provisional application No. 62/198,215, filed on Jul. 29, 2015.

(51) Int. Cl. C07C 211/61; C07D 209/88; C07D 307/91; H01L 51/50; C09K 11/06

(52) U.S. Cl. CPC C07C 211/61; H01L 51/5012; C09K 11/06; C07D 307/91; C07D 209/88; C09K 2211/1011; C09K 2211/1014; C09K 2211/1088; C09K 2211/1022; C09K 2211/1029; C07C 2103/50

(57) ABSTRACT

There is provided a compound having Formulae I-IV:

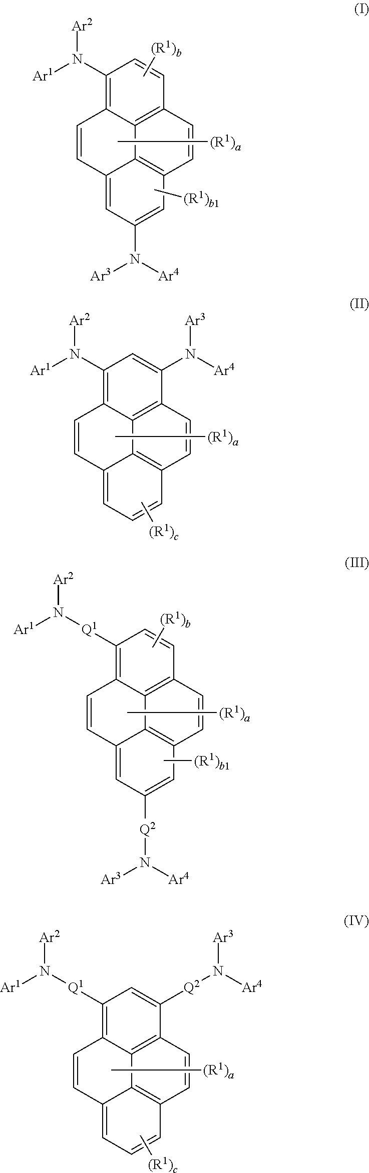

In the formulae: Q¹ and Q² are the same or different and are a single bond, hydrocarbon aryl, or deuterated hydrocarbon aryl; Ar¹-Ar⁴ are the same or different and are hydrocarbon aryl, heteroaryl, substituted derivatives thereof, or deuterated analogs thereof, where Ar¹ and Ar² may be joined to form a carbazole group and Ar³ and Ar⁴ may be joined to form a carbazole group; R¹ is the same or different at each occurrence and is D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, or deuterated germyl; a is an integer of 0-4; b and b1 are the same or different and are an integer of 0-2; and c is an integer of 0-3.

### CLAIM OF BENEFIT OF PRIOR APPLICATION

This application claims the benefit of U.S. Provisional Application No. 62/198,215, filed Jul. 29, 2015, which is incorporated in its entirety herein by reference.

### BACKGROUND INFORMATION

Field of the Disclosure

This disclosure relates in general to luminescent compounds and their use in electronic devices.

Description of the Related Art

Organic electronic devices that emit light, such as light-emitting diodes that make up displays, are present in many different kinds of electronic equipment. In all such devices, an organic active layer is sandwiched between two electrical contact layers. At least one of the electrical contact layers is light-transmitting so that light can pass through the electrical contact layer. The organic active layer emits light through the light-transmitting electrical contact layer upon application of electricity across the electrical contact layers.

It is well known to use organic electroluminescent compounds as the active component in light-emitting diodes. Simple organic molecules, such as anthracene, thiadiazole derivatives, and coumarin derivatives are known to show electroluminescence. Metal complexes, particularly iridium and platinum complexes are also known to show electroluminescence. In some cases these small molecule compounds are present as a dopant in a host material to improve processing and/or electronic properties.

There is a continuing need for new luminescent compounds.

### SUMMARY

There is provided a compound having Formula I, as described below in the detailed description.

There is provided a compound having Formula II, as described below in the detailed description.

There is provided a compound having Formula III, as described below in the detailed description.

There is provided a compound having Formula IV, as described below in the detailed description.

There is also provided an organic electronic device comprising a first electrical contact, a second electrical contact and a photoactive layer therebetween, the photoactive layer comprising a compound having Formulae I-IV.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 includes an illustration of one example of an organic electronic device including a new compound described herein.

FIG. 2 includes an illustration of another example of an organic electronic device including a new compound described herein.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description first addresses Definitions and Clarification of Terms followed by the Compound Having Formula I, the Compound Having Formula II, the Compound Having Formula III, the Compound Having Formula IV, Devices, and finally Examples.

### 1. Definitions and Clarification of Terms

Before addressing details of embodiments described below, some terms are defined or clarified.

As used in the “Definitions and Clarification of Terms”, R, R′ and R″ and any other variables are generic designations and may be the same as or different from those defined in the formulas.

The term “adjacent” as it refers to substituent groups refers to groups that are bonded to carbons that are joined together with a single or multiple bond. Exemplary adjacent R groups are shown below:

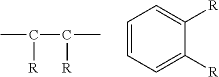

The term “alkoxy” is intended to mean the group RO—, where R is an alkyl group.

The term “alkyl” is intended to mean a group derived from an aliphatic hydrocarbon and includes a linear, a branched, or a cyclic group. In some embodiments, an alkyl has from 1-20 carbon atoms.

The term “aromatic compound” is intended to mean an organic compound comprising at least one unsaturated cyclic group having 4n+2 delocalized pi electrons.

The term “aryl” is intended to mean a group derived from an aromatic hydrocarbon having one point of attachment. The term includes groups which have a single ring and those which have multiple rings which can be joined by a single bond or fused together. Hydrocarbon aryl groups have only carbon in the ring structures. Heteroaryl groups have at least one heteroatom in a ring structure. The term “alkylaryl” is intended to mean an aryl group having one or more alkyl substituents.

The term “aryloxy” is intended to mean the group RO—, where R is an aryl group.

The term “charge transport,” when referring to a layer, material, member, or structure is intended to mean such layer, material, member, or structure facilitates migration of such charge through the thickness of such layer, material, member, or structure with relative efficiency and small loss of charge. Hole transport materials facilitate positive charge; electron transport materials facilitate negative charge. Although light-emitting materials may also have some charge transport properties, the term “charge transport layer, material, member, or structure” is not intended to include a layer, material, member, or structure whose primary function is light emission.

The term “deuterated” is intended to mean that at least one hydrogen (“H”) has been replaced by deuterium (“D”). The term “deuterated analog” refers to a structural analog of a compound or group in which one or more available hydrogens have been replaced with deuterium. In a deuterated compound or deuterated analog, the deuterium is present in at least 100 times the natural abundance level. The term “Vo deuterated” or “Vo deuteration” is intended to mean the ratio of deuterons to the sum of protons plus deuterons, expressed as a percentage.

The term “dopant” is intended to mean a material, within a layer including a host material, that changes the electronic characteristic(s) or the targeted wavelength(s) of radiation emission, reception, or filtering of the layer compared to the electronic characteristic(s) or the wavelength(s) of radiation emission, reception, or filtering of the layer in the absence of such material.

The term “germyl” refers to the group R₃Ge—, where R is the same or different at each occurrence and is H, D, C1-20 alkyl, deuterated alkyl, fluoroalkyl, aryl, or deuterated aryl. In some embodiments, one or more carbons in an R alkyl group are replaced with Ge.

The prefix “hetero” indicates that one or more carbon atoms have been replaced with a different atom. In some embodiments, the different atom is N, O, or S.

The term “host material” is intended to mean a material, usually in the form of a layer, to which a dopant may be added. The host material may or may not have electronic characteristic(s) or the ability to emit, receive, or filter radiation.

The terms “luminescent material”, “emissive material” and “emitter” are intended to mean a material that emits light when activated by an applied voltage (such as in a light-emitting diode or light-emitting electrochemical cell). The term “blue luminescent material” is intended to mean a material capable of emitting radiation that has an emission maximum at a wavelength in a range of approximately 445-490 nm.

The term “layer” is used interchangeably with the term “film” and refers to a coating covering a desired area. The term is not limited by size. The area can be as large as an entire device or as small as a specific functional area such as the actual visual display, or as small as a single sub-pixel. Layers and films can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, and continuous nozzle coating or printing. Discontinuous deposition techniques include, but are not limited to, ink jet printing, gravure printing, and screen printing.

The term “organic electronic device” or sometimes just “electronic device” is intended to mean a device including one or more organic semiconductor layers or materials.

The term “photoactive” refers to a material or layer that emits light when activated by an applied voltage (such as in a light emitting diode or chemical cell), that emits light after the absorption of photons (such as in down-converting phosphor devices), or that responds to radiant energy and generates a signal with or without an applied bias voltage (such as in a photodetector or a photovoltaic cell).

The term “siloxane” refers to the group R₃SiOR₂Si—, where R is the same or different at each occurrence and is H, D, C1-20 alkyl, deuterated alkyl, fluoroalkyl, aryl, or deuterated aryl. In some embodiments, one or more carbons in an R alkyl group are replaced with Si.

The term “siloxy” refers to the group R₃SiO—, where R is the same or different at each occurrence and is H, D, C1-20 alkyl, deuterated alkyl, fluoroalkyl, aryl, or deuterated aryl.

The term “silyl” refers to the group R₃Si—, where R is the same or different at each occurrence and is H, D, C1-20 alkyl, deuterated alkyl, fluoroalkyl, aryl, or deuterated aryl. In some embodiments, one or more carbons in an R alkyl group are replaced with Si.

A group “derived from” a compound, indicates the radical formed by removal of one H or D.

All groups may be unsubstituted or substituted. The substituent groups are discussed below. In a structure where a substituent bond passes through one or more rings as shown below,

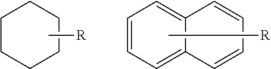

it is meant that the substituent R may be bonded at any available position on the one or more rings.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the disclosed subject matter hereof, is described as consisting essentially of certain features or elements, in which embodiment features or elements that would materially alter the principle of operation or the distinguishing characteristics of the embodiment are not present therein. A further alternative embodiment of the described subject matter hereof is described as consisting of certain features or elements, in which embodiment, or in insubstantial variations thereof, only the features or elements specifically stated or described are present.

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Group numbers corresponding to columns within the Periodic Table of the elements use the “New Notation” convention as seen in the CRC Handbook of Chemistry and Physics, 81st Edition (2000-2001).

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. In addition, the materials, methods, and examples are illustrative only and not intended to be limiting.

To the extent not described herein, many details regarding specific materials, processing acts, and circuits are conventional and may be found in textbooks and other sources within the organic light-emitting diode display, photodetector, photovoltaic cell, and semiconductive member arts.

### 2. Compounds Having Formula I

In some embodiments, the compounds described herein have

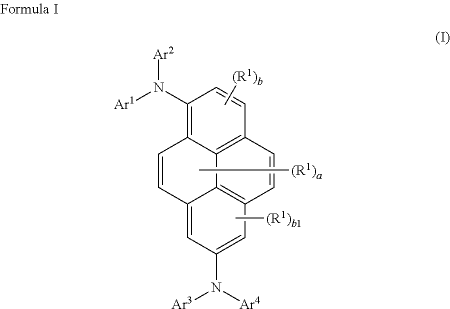

wherein:
Ar¹-Ar⁴ are the same or different and are selected from the group consisting of hydrocarbon aryl, heteroaryl, substituted derivatives thereof, and deuterated analogs thereof, wherein Ar¹ and Ar² may be joined to form a carbazole group and Ar³ and Ar⁴ may be joined to form a carbazole group;
R¹ is the same or different at each occurrence and is selected from the group consisting of D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, and deuterated germyl;
a is an integer of 0-4; and
b and b1 are the same or different and are an integer of 0-2.

In some embodiments, the compounds having Formula I are useful as emissive materials. In some embodiments, the compounds are blue emissive materials. They can be used alone or as a dopant in a host material.

In some embodiments, compounds having Formula I have an unexpectedly narrow emission profile. In some embodiments, the emission profile has a width at half the maximum intensity (“FWHM”) that is less than 75 nm; in some embodiments, less than 60 nm; in some embodiments, less than 50 nm. This is advantageous for display devices for producing more saturated color.

In some embodiments, the compounds having Formula I have deep blue color. As used herein, the term “deep blue color” refers to a c.i.e. y-coordinate of less than 0.10, according to the C.I.E. chromaticity scale (Commission Internationale de L'Eclairage, 1931).

In some embodiments, the compounds having Formula I have a photoluminescence y-coordinate of less than 0.10; in some embodiments, less than 0.090.

In some embodiments, devices including the compounds of Formula I have improved efficiencies. In some embodiments, the efficiency of a device including Formula I is greater than 4.5 cd/A at 1000 nits; in some embodiments, greater than 5.0 cd/A at 1000 nits.

In some embodiments, devices including the compounds of Formula I have increased lifetime. In some embodiments, devices including the compounds of Formula I have a T70 greater than 1000 hours at 50° C. As used herein, T70 refers to the time to reach 70% of initial luminance. In some embodiments, devices including the compounds of Formula I have a T70 greater than 1500 hours at 50° C.

In some embodiments, electroluminescent devices including the compounds of Formula I as emissive materials have deep blue color. In some embodiments, the x-coordinate is less than 0.15 and the y-coordinate is less than 0.10; in some embodiments, the y-coordinate is less than 0.090.

In some embodiments of Formula I, the compound is deuterated. In some embodiments, the compound is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments of Formula I, deuteration is present on the core pyrene group.

In some embodiments of Formula I, deuteration is present on one or more substituent groups.

In some embodiments of Formula I, deuteration is present on the core pyrene group and one or more substituent groups.

In some embodiments of Formula I, the compound has no carbazole groups, substituted derivatives, or deuterated analogs thereof.

In some embodiments of Formula I, Ar¹ is an aryl group having 6-36 ring carbons or deuterated analog thereof. The aryl group can include one or more single ring groups bonded together, one or more fused rings, or combinations thereof.

In some embodiments of Formula I, Ar¹ has no heteroaromatic groups.

In some embodiments of Formula I, Ar¹ includes no hydrocarbon aryl groups with more than two fused rings.

In some embodiments of Formula I, Ar¹ includes no hydrocarbon aryl groups with fused rings.

In some embodiments of Formula I, Ar¹ is an aryl group having no additional substituents.

In some embodiments of Formula I, Ar¹ is an aryl group having at least one substituent selected from the group consisting of D, F, alkyl, fluoroalkyl, alkoxy, siloxane, silyl, germyl, diarylamino, N-heteroaryl, N,O-heteroaryl, N,S-heteroaryl, deuterated alkyl, deuterated fluoroalkyl, deuterated alkoxy, deuterated siloxane, deuterated silyl, deuterated germyl, deuterated diarylamino, and deuterated N-heteroaryl, deuterated N,O-heteroaryl, and deuterated N,S-heteroaryl.

In some embodiments of Formula I, Ar¹ has Formula a

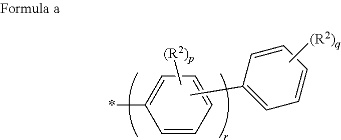

where:
R² is the same or different at each occurrence and is selected from the group consisting of D, F, alkyl, fluoroalkyl, alkoxy, siloxane, silyl, germyl, diarylamino, N-heteroaryl, N,O-heteroaryl, N,S-heteroaryl, deuterated alkyl, deuterated fluoroalkyl, deuterated alkoxy, deuterated siloxane, deuterated silyl, deuterated germyl, deuterated diarylamino, and deuterated N-heteroaryl, deuterated N,O-heteroaryl, and deuterated N,S-heteroaryl, where adjacent R² groups can be joined together to form an fused aromatic ring or a deuterated fused aromatic ring;
p is the same or different at each occurrence and is an integer from 0-4;
q is an integer from 0-5;
r is an integer from 1 to 5; and
* indicates the point of attachment.

In some embodiments of Formula I, Ar¹ has Formula b

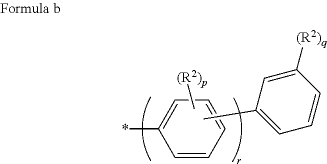

where R², p, q, r and * are as in Formula a.

In some embodiments of Formula I, Ar¹ is selected from the group consisting of phenyl, biphenyl, terphenyl, napthyl, naphthylphenyl, phenylnaphthyl, styryl, derivatives thereof having one or more substituents selected from the group consisting of fluoro, alkyl, alkoxy, silyl, siloxy, and deuterated analogs thereof.

In some embodiments of Formula I, Ar¹ has at least one substituent that is an N-heteroaryl or deuterated N-heteroaryl having at least one ring atom which is N.

In some embodiments of Formula I, Ar¹ is a hydrocarbon aryl and has at least one substituent that is an N-heteroaryl or deuterated N-heteroaryl having at least one ring atom which is N.

In some embodiments, the N-heteroaryl is derived from a compound selected from the group consisting of pyrrole, pyridine, pyrimidine, carbazole, imidazole, benzimidazole, imidazolobenzimidazole, triazole, benzotriazole, triazolopyridine, indolocarbazole, indole, indoloindole, phenanthroline, quinoline, isoquinoline, quinoxaline, substituted derivatives thereof, and deuterated analogs thereof.

In some embodiments, the N-heteroaryl is derived from a compound selected from the group consisting of pyrrole, pyridine, pyrimidine, indolocarbazole, indole, indoloindole, phenanthroline, quinoline, isoquinoline, substituted derivatives thereof, and deuterated analogs thereof. In some embodiments of the substituted derivatives, the substituent is selected from the group consisting of D, alkyl, silyl, aryl, deuterated alkyl, deuterated silyl, and deuterated aryl.

In some embodiments, the N-heteroaryl is derived from a carbazole or deuterated carbazole.

In some embodiments, the N-heteroaryl is derived from a carbazole or deuterated carbazole and has formula Cz-1:

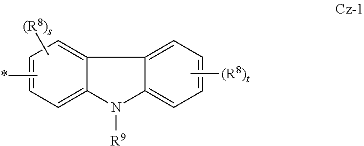

wherein:
R⁸ is the same or different at each occurrence and is selected from the group consisting of D, alkyl, silyl, aryl, deuterated alkyl, deuterated silyl, and deuterated aryl;
R⁹ is selected from the group consisting of aryl and deuterated aryl;
s is an integer of 0-3;
t is an integer of 0-4; and
* represents the point of attachment.

In some embodiments, the N-heteroaryl is derived from a carbazole or deuterated carbazole and has formula Cz-2:

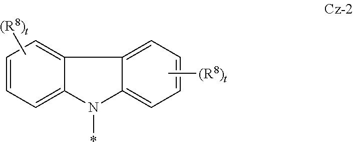

where R⁸, t, and * are as defined above for Cz-1.

In some embodiments, the N-heteroaryl is derived from a carbazole or deuterated carbazole and has formula Cz-3:

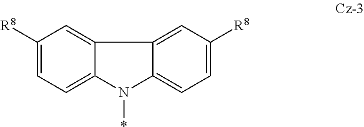

where R⁸ and * are as defined above for Cz-1.

In some embodiments, the N-heteroaryl is derived from a carbazole or deuterated carbazole and has formula Cz-4:

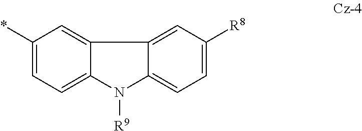

where R⁸, R⁹ and * are as defined above for Cz-1.

In some embodiments, the N-heteroaryl is derived from a carbazole or deuterated carbazole and has formula Cz-5:

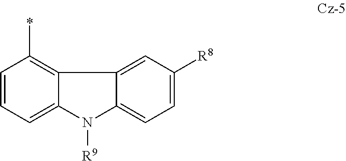

where R⁸, R⁹ and * are as defined above for Cz-1.

In some embodiments, the N-heteroaryl is derived from a benzimidazole or deuterated benzimidazole and has formula BzI-1:

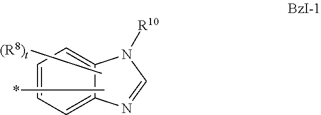

where R¹⁰ is selected from the group consisting of alkyl, silyl, aryl, and deuterated analogs thereof; R⁸ and *are as defined above for Cz-1.

In some embodiments, the N-heteroaryl is derived from a benzimidazole or deuterated benzimidazole and has formula BzI-2:

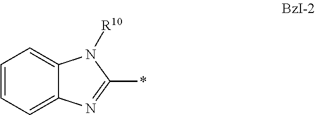

where R¹⁰ and * are as defined above for BzI-1.

In some embodiments of Formula I, Ar¹ has at least one substituent that is an N,O-heteroaryl having at least one ring atom that is N and at least one ring atom that is O.

In some embodiments of Formula I, Ar¹ is a hydrocarbon aryl and has at least one substituent that is an N,O-heteroaryl having at least one ring atom that is N and at least one ring atom that is O.

In some embodiments, the N,O-heteroaryl is derived from a compound selected from the group consisting of oxazole, benzoxazole, oxazine, benzoxazine, dibenzoxazine, and deuterated analogs thereof.

In some embodiments, the N,O-heteroaryl is derived from a benzoxazole or deuterated benzoxazole and has formula BzO-1:

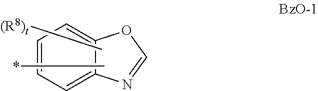

where t, R⁸, and * are as defined above for Cz-1.

In some embodiments, the N,O-heteroaryl is derived from a benzoxazole or deuterated benzoxazole and has formula BzO-2:

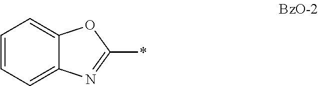 [SMILES: *c1nc2ccccc2o1]

where * represents the point of attachment.

In some embodiments, the N,O-heteroaryl is derived from a dibenzoxazine or deuterated dibenzoxazine and has formula DBO-1

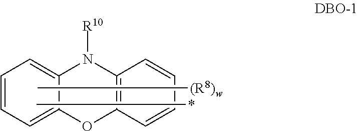

where w=0-7, R⁸ is as defined above for Cz-1, R¹⁰ and * are as defined above for BzI-1.

In some embodiments, the N,O-heteroaryl is derived from a dibenzoxazine or deuterated dibenzoxazine and has formula DBO-2

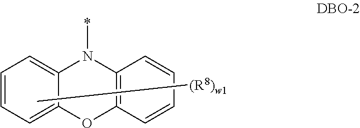

where w1=0-8, and R⁸ and * are as defined above for Cz-1.

In some embodiments of Formula I, Ar¹ has at least one substituent that is an N,S-heteroaryl having at least one ring atom that is N and at least one ring atom that is S.

In some embodiments of Formula I, Ar¹ is a hydrocarbon aryl and has at least one substituent that is an N,S-heteroaryl having at least one ring atom that is N and at least one ring atom that is S.

In some embodiments, the N,S-heteroaryl is derived from a compound selected from the group consisting of thiazole, benzothiazole, and deuterated analogs thereof.

In some embodiments, the N,S-heteroaryl is derived from a benzothiazole or deuterated benzothiazole and has formula BT-1:

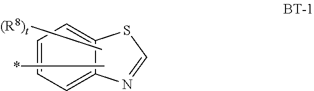

where t, R⁸ and * are as defined above for Cz-1.

In some embodiments, the N,S-heteroaryl is derived from a benzothiazole or deuterated benzothiazole and has formula BT-2:

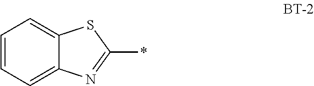 [SMILES: *c1nc2ccccc2s1]

where * represents the point of attachment.

In some embodiments of Formula I, Ar¹ has at least one substituent that is an O-heteroaryl having at least one ring atom that is O.

In some embodiments of Formula I, Ar¹ is a hydrocarbon aryl and has at least one substituent that is an O-heteroaryl having at least one ring atom that is O.

In some embodiments, the O-heteroaryl is derived from a compound selected from the group consisting of furan, benzofuran, dibenzofuran, substituted derivatives thereof, and deuterated analogs thereof.

In some embodiments, the O-heteroaryl is a dibenzofuran or deuterated dibenzofuran.

In some embodiments, the O-heteroaryl is a dibenzofuran or deuterated dibenzofuran having formula DBF-1:

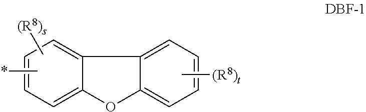

where R⁸, R⁹ and * are as defined above for Cz-1.

In some embodiments, the O-heteroaryl is a dibenzofuran or deuterated dibenzofuran having formula DBF-2:

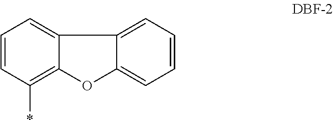 [SMILES: *c1cccc2c1oc1ccccc12]

where * represents the point of attachment.

In some embodiments, the O-heteroaryl is a dibenzofuran or deuterated dibenzofuran having formula DBF-3:

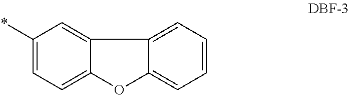 [SMILES: *c1ccc2oc3ccccc3c2c1]

where * represents the point of attachment.

In some embodiments of Formula I, Ar¹ has at least one substituent that is an S-heteroaryl having at least one ring atom that is S.

In some embodiments of Formula I, Ar¹ is a hydrocarbon aryl and has at least one substituent that is an S-heteroaryl having at least one ring atom that is S.

In some embodiments, the S-heteroaryl is derived from a compound selected form the group consisting of thiophene, benzothiophene, dibenzothiophene, substituted derivatives thereof, and deuterated analogs thereof.

In some embodiments, the S-heteroaryl is a dibenzothiophene or deuterated dibenzothiophene.

In some embodiments, the S-heteroaryl is a dibenzothiophene or deuterated dibenzothiophene having formula DBT-1

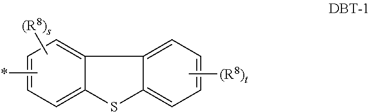

where R⁸, R⁹ and * are as defined above for Cz-1.

In some embodiments, the S-heteroaryl is a dibenzothiophene or deuterated dibenzothiophene having formula DBT-2:

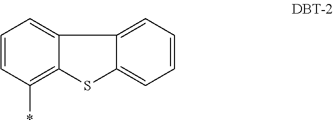 [SMILES: *c1cccc2c1sc1ccccc12]

where * represents the point of attachment.

In some embodiments, the S-heteroaryl is a dibenzothiophene or deuterated dibenzothiophene having formula DBT-3:

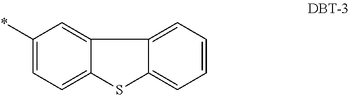 [SMILES: *c1ccc2sc3ccccc3c2c1]

wherein * represents the point of attachment.

In some embodiments of Formula I, Ar¹ is a heteroaryl or deuterated heteroaryl.

In some embodiments of Formula I, Ar¹ is an N-heteroaryl, as described above.

In some embodiments of Formula I, Ar¹ is an N,O-heteroaryl, as described above.

In some embodiments of Formula I, Ar¹ is an N,S-heteroaryl, as described above.

In some embodiments of Formula I, Ar¹ is an O-heteroaryl, as described above.

In some embodiments of Formula I, Ar¹ is an S-heteroaryl, as described above.

In Formula I, all of the above embodiments for Ar¹ apply equally to Ar².

In Formula I, all of the above embodiments for Ar¹ apply equally to Ar³.

In Formula I, all of the above embodiments for Ar¹ apply equally to Ar⁴.

In some embodiments of Formula I, Ar¹═Ar³.

In some embodiments of Formula I, Ar²═Ar⁴.

In some embodiments of Formula I, Ar¹ ≠ Ar².

In some embodiments of Formula I, Ar³ ≠ Ar⁴.

In some embodiments of Formula I, Ar¹═Ar²═Ar³═Ar⁴.

In some embodiments of Formula I, Ar¹ ≠ Ar² ≠ Ar³ ≠ Ar⁴.

In some embodiments of Formula I, the compounds have differently-substituted amino groups. By this it is meant that the —NAr¹Ar² substituent is different from the —NAr³Ar⁴ substituent.

Compounds having Formula (I) and having differently-substituted amino groups may be prepared in a number of different ways by one skilled in the art. For example, such embodiments can be prepared starting from trimethyl-[7-(4,4,5,5-tetramethyl-1,3,2-dioxaborolan-2-yl)pyren-1-yl]silane (Intermediate 5) as shown in Scheme 1. The synthesis of Intermediate 5 is provided in the Examples.

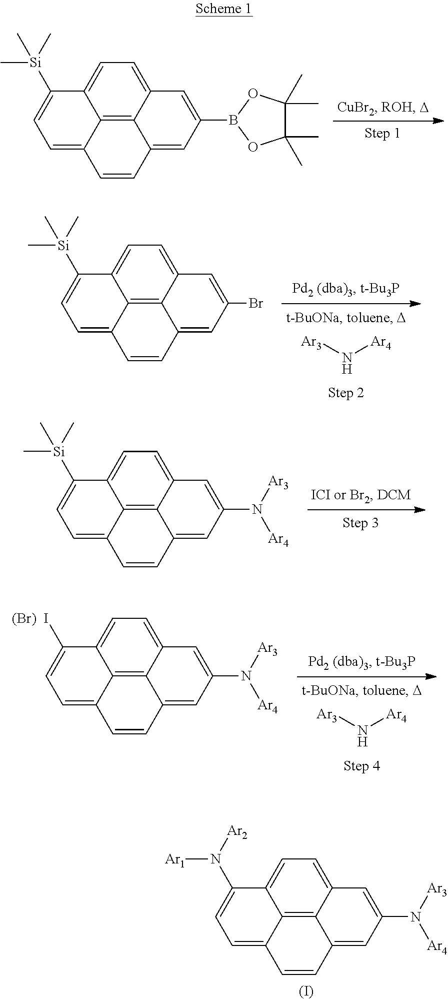

Treatment of Intermediate 5 with alcoholic copper(II) bromide, with co-solvent as needed, provides (7-bromopyren-1-yl)-trimethylsilane which is aminated with the diarylamine Ar³(Ar⁴)NH under palladium catalyzed conditions in Step 2. Halo-desilylation with iodine monochloride or N-bromosuccinimide (NBS) as shown in Step 3 gives the 1-iodo- or 1-bromo-substrate, respectively, for the final palladium-catalyzed amination with the diarylamine Ar¹(Ar²)NH in Step 4 to provide the product of Formula I having differently-substituted amino groups.

In some embodiments of Formula I, a=0.

In some embodiments of Formula I, a=1.

In some embodiments of Formula I, a=2.

In some embodiments of Formula I, a=3.

In some embodiments of Formula I, a=4.

In Formula I, R¹ has no amino groups.

In some embodiments of Formula I, R¹ has no heteroaromatic groups.

In some embodiments of Formula I, R¹ has no substituent groups.

In some embodiments of Formula I, a>0 and at least one R¹ is an alkyl or deuterated alkyl having 1-20 carbons; in some embodiments, 1-12 carbons; in some embodiments, 3-8 carbons.

In some embodiments of Formula I, a>0 and at least one R¹ is a hydrocarbon aryl group having 6-36 ring carbons. The hydrocarbon aryl group can include one or more single ring groups bonded together, one or more fused rings, or combinations thereof.

In some embodiments of Formula I, a>0 and at least one R¹ has Formula a1

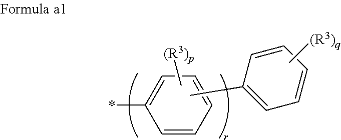

where:
R³ is the same or different at each occurrence and is selected from the group consisting of D, F, alkyl, fluoroalkyl, alkoxy, siloxane, silyl, germyl, deuterated alkyl, deuterated fluoroalkyl, deuterated alkoxy, deuterated siloxane, deuterated silyl, and deuterated germyl, where adjacent R³ groups can be joined together to form an fused aromatic ring or a deuterated fused aromatic ring;
p is the same or different at each occurrence and is an integer from 0-4;
q is an integer from 0-5;
r is an integer from 1 to 5; and
* indicates the point of attachment.

In some embodiments of Formula I, a>0 and at least one R¹ has Formula b1

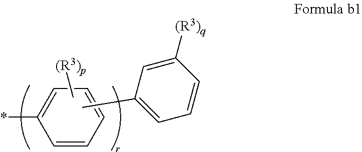

where R³, p, q, r and * are as in Formula a1.

In some embodiments of Formula I, b=0.

In some embodiments of Formula I, b=1.

In some embodiments of Formula I, b=2.

In some embodiments of Formula I, b>0 and at least one R¹ is as described above.

In some embodiments of Formula I, b1=0.

In some embodiments of Formula I, b1=1.

In some embodiments of Formula I, b1=2.

In some embodiments of Formula I, b1>0 and at least one R¹ is as described above.

Any of the above embodiments of Formula I can be combined with one or more of the other embodiments, so long as they are not mutually exclusive. For example, the embodiment in which Ar¹ has at least one substituent that is an N,O-heteroaryl can be combined with the embodiment in which a=1 and at least one R¹ is a hydrocarbon aryl. The same is true for the other non-mutually-exclusive embodiments discussed above. The skilled person would understand which embodiments were mutually exclusive and would thus readily be able to determine the combinations of embodiments that are contemplated by the present application.

The compounds of Formula I can be made using any technique that will yield a C—C or C—N bond. A variety of such techniques are known, such as Suzuki, Stille, and metal-catalyzed C—N couplings as well as metal catalyzed and oxidative direct arylation.

Deuterated compounds can be prepared in a similar manner using deuterated precursor materials or, more generally, by treating the non-deuterated compound with deuterated solvent, such as benzene-d6, in the presence of a Bronsted acid H/D exchange catalyst, such as trifluoromethanesulfonic acid, or a Lewis acid H/D exchange catalyst, such as aluminum trichloride or ethyl aluminum dichloride.

Exemplary preparations are given in the Examples.

Examples of compounds having Formula I include, but are not limited to, the compounds shown below.

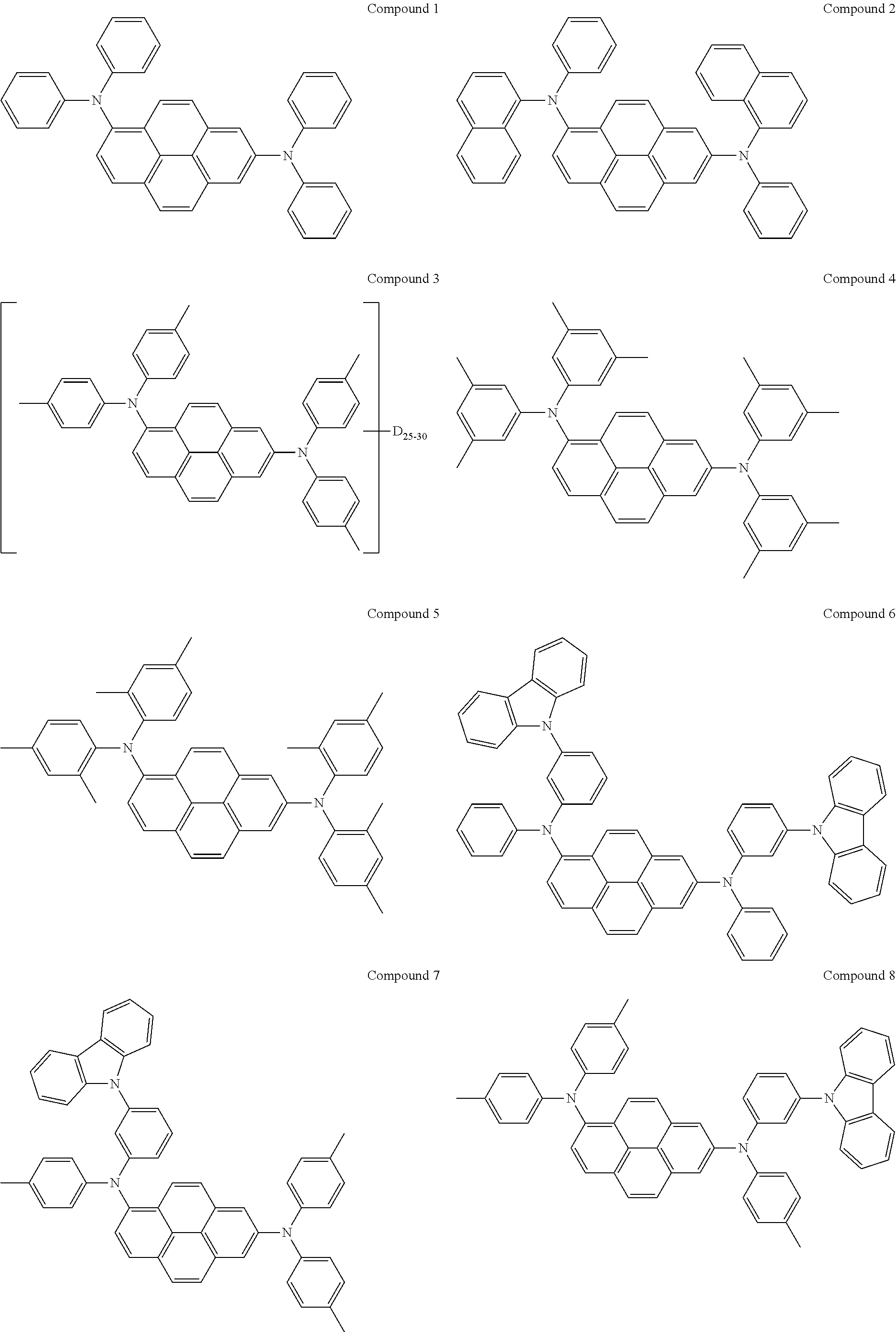

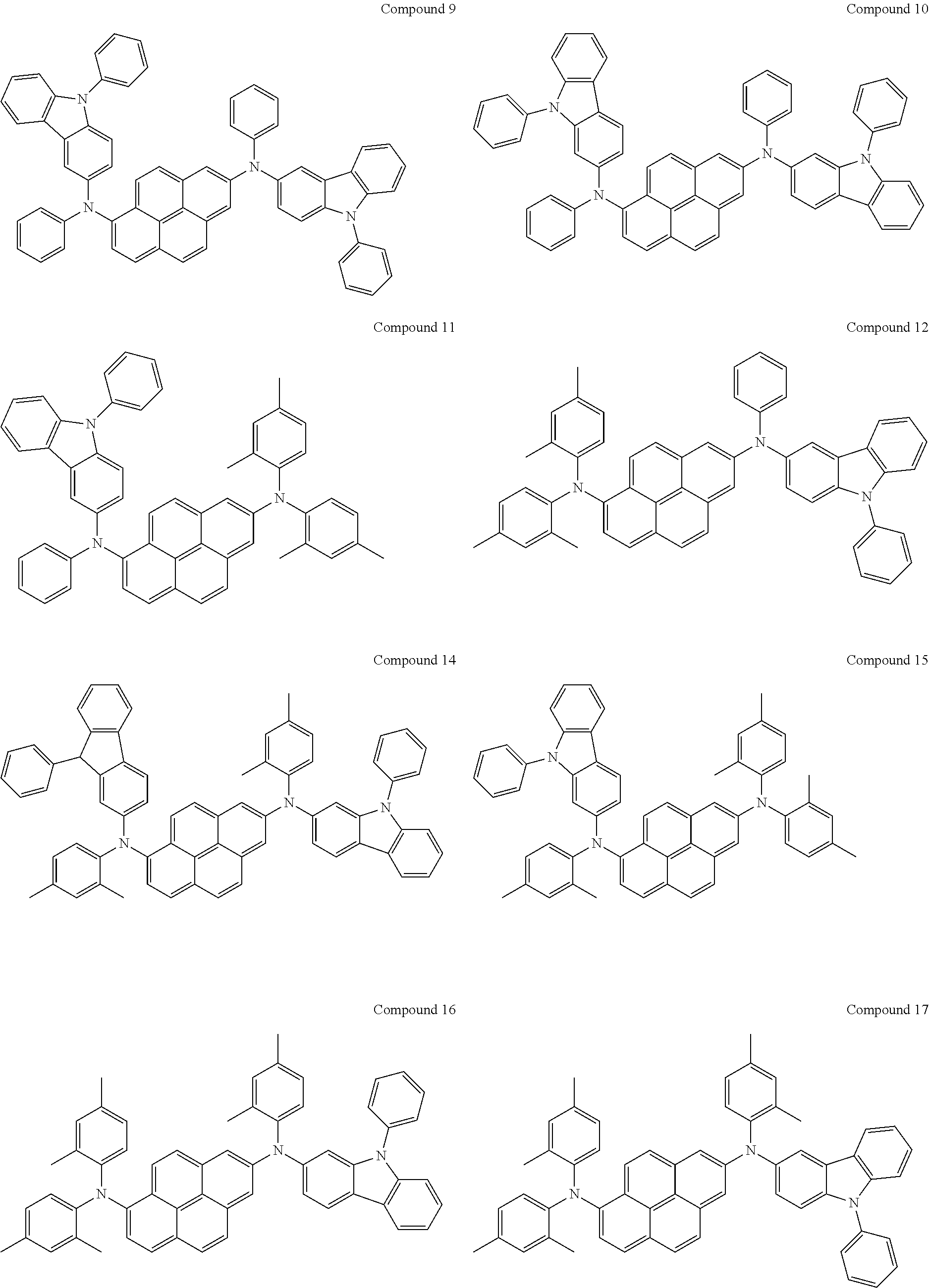

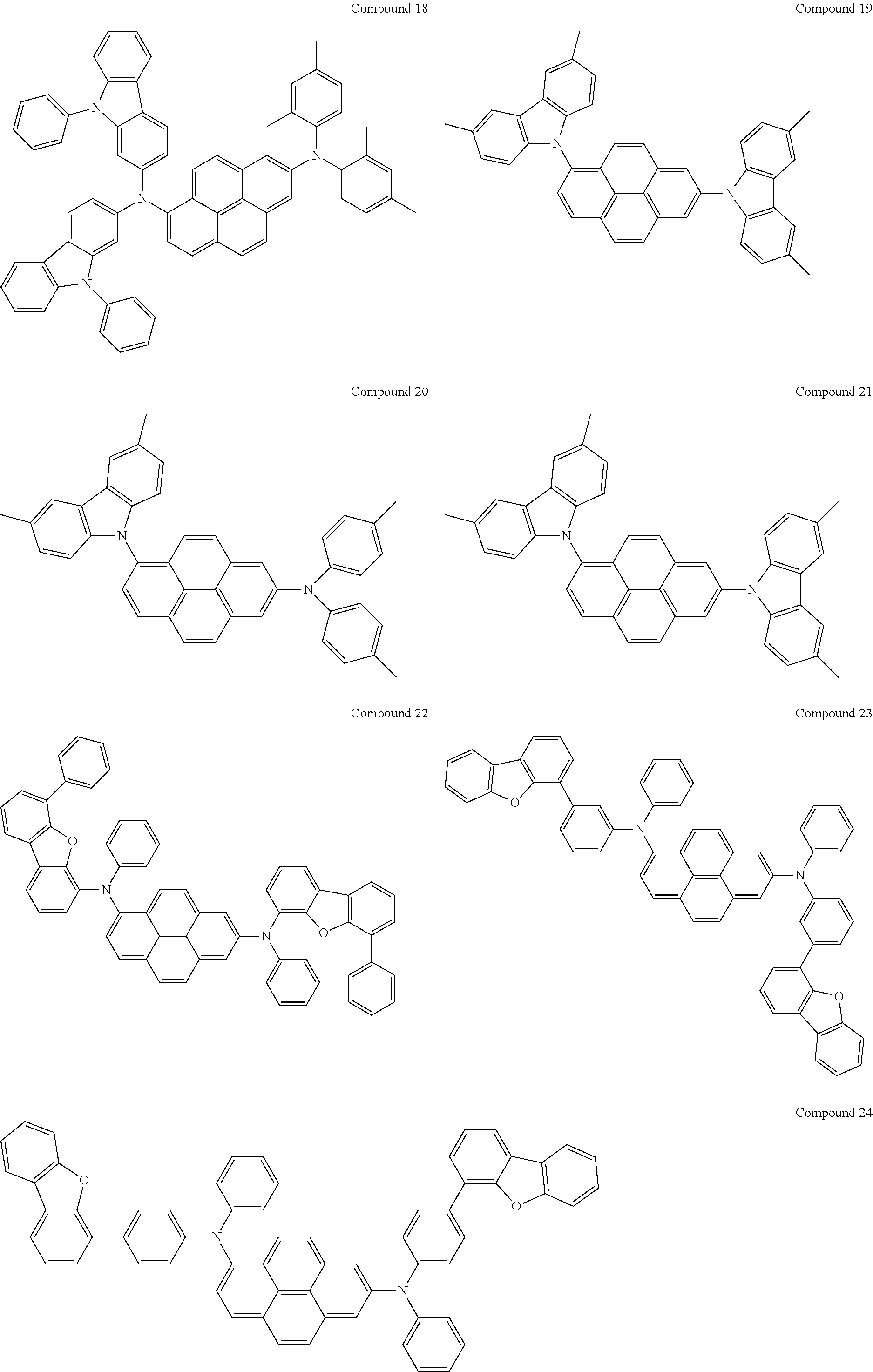

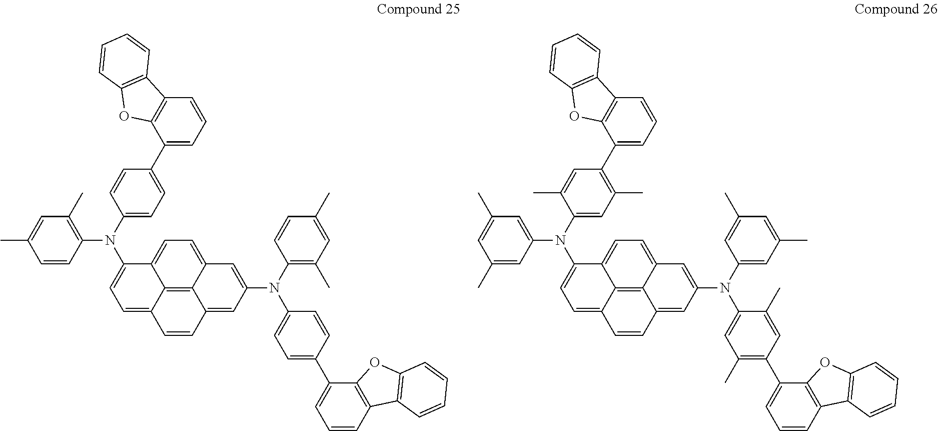 [SMILES: Cc1ccc(N(c2ccc(-c3cccc4c3oc3ccccc34)cc2)c2cc3ccc4ccc(N(c5ccc(-c6cccc7c6oc6ccccc67)cc5)c5ccc(C)cc5C)c5ccc(c2)c3c45)c(C)c1 ; Cc1cc(C)cc(N(c2cc3ccc4ccc(N(c5cc(C)cc(C)c5)c5cc(C)c(-c6cccc7c6oc6ccccc67)cc5C)c5ccc(c2)c3c45)c2cc(C)c(-c3cccc4c3oc3ccccc34)cc2C)c1]

### 3. Compounds Having Formula II

In some embodiments, the compounds described herein have Formula II

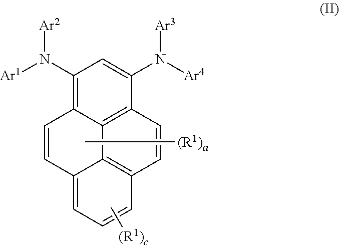

wherein:
Ar¹-Ar⁴ are the same or different and are selected from the group consisting of hydrocarbon aryl, heteroaryl, substituted derivatives thereof, and deuterated analogs thereof, wherein Ar¹ and Ar² may be joined to form a carbazole group and Ar³ and Ar⁴ may be joined to form a carbazole group;
R¹ is the same or different at each occurrence and is selected from the group consisting of D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, and deuterated germyl;
a is an integer of 0-4; and
c is an integer of 0-3.

In some embodiments, the compounds having Formula II are useful as emissive materials. In some embodiments, the compounds are blue emissive materials. They can be used alone or as a dopant in a host material.

In some embodiments, compounds having Formula II have an unexpectedly narrow emission profile. In some embodiments, the emission profile has a width at half the maximum intensity (“FWHM”) that is less than 75 nm; in some embodiments, less than 60 nm; in some embodiments, less than 50 nm. This is advantageous for display devices for producing more saturated color.

In some embodiments, the compounds having Formula II have deep blue color. As used herein, the term “deep blue color” refers to a c.i.e. y-coordinate of less than 0.10, according to the C.I.E. chromaticity scale (Commission Internationale de L'Eclairage, 1931). In some embodiments, the compounds having Formula II have a photoluminescence y-coordinate of less than 0.10; in some embodiments, less than 0.090.

In some embodiments, devices including the compounds of Formula II have improved efficiencies. In some embodiments, the efficiency of a device including Formula II is greater than 4.5 cd/A at 1000 nits; in some embodiments, greater than 5.0 cd/A at 1000 nits.

In some embodiments, devices including the compounds of Formula II have increased lifetime. In some embodiments, devices including the compounds of Formula II have a T70 greater than 1000 hours at 50° C. As used herein, T70 refers to the time to reach 70% of initial luminance. In some embodiments, devices including the compounds of Formula II have a T70 greater than 1500 hours at 50° C.

In some embodiments, electroluminescent devices including the compounds of Formula II as emissive materials have deep blue color. In some embodiments, the x-coordinate is less than 0.15 and the y-coordinate is less than 0.10; in some embodiments, the y-coordinate is less than 0.090.

In some embodiments of Formula II, the compound is deuterated. In some embodiments, the compound is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments of Formula II, deuteration is present on the core pyrene group.

In some embodiments of Formula II, deuteration is present on one or more substituent groups.

In some embodiments of Formula II, deuteration is present on the core pyrene group and one or more substituent groups.

All of the embodiments for Ar¹ described above for Formula I, apply equally to Formula II.

All of the embodiments for Ar² described above for Formula I, apply equally to Formula II.

All of the embodiments for Ar³ described above for Formula I, apply equally to Formula II.

All of the embodiments for Ar⁴ described above for Formula I, apply equally to Formula II.

In some embodiments of Formula II, the compounds have differently-substituted amino groups.

In some embodiments of Formula II, c=0.

In some embodiments of Formula II, c=1.

In some embodiments of Formula II, c=2.

In some embodiments of Formula II, c=3.

In some embodiments of Formula II, c>0.

All of the embodiments for a and R¹ described above for Formula I, apply equally to Formula II.

Any of the above embodiments of Formula II can be combined with one or more of the other embodiments, so long as they are not mutually exclusive. For example, the embodiment in which Ar¹ has a substituent that is an N,O-heteroaryl can be combined with the embodiment in which a=1 and at least one R¹ is a hydrocarbon aryl. The same is true for the other non-mutually-exclusive embodiments discussed above. The skilled person would understand which embodiments were mutually exclusive and would thus readily be able to determine the combinations of embodiments that are contemplated by the present application.

The compounds of Formula II can be made using any technique that will yield a C—C or C—N bond. A variety of such techniques are known, such as Suzuki, Stille, and metal-catalyzed C—N couplings as well as metal catalyzed and oxidative direct arylation.

Deuterated compounds can be prepared in a similar manner using deuterated precursor materials or, more generally, by treating the non-deuterated compound with deuterated solvent, such as benzene-d6, in the presence of a Bronsted acid H/D exchange catalyst, such as trifluoromethanesulfonic acid, or a Lewis acid H/D exchange catalyst, such as aluminum trichloride or ethyl aluminum dichloride.

Exemplary preparations are given in the Examples.

Examples of compounds having Formula II include, but are not limited to, the compounds shown below.

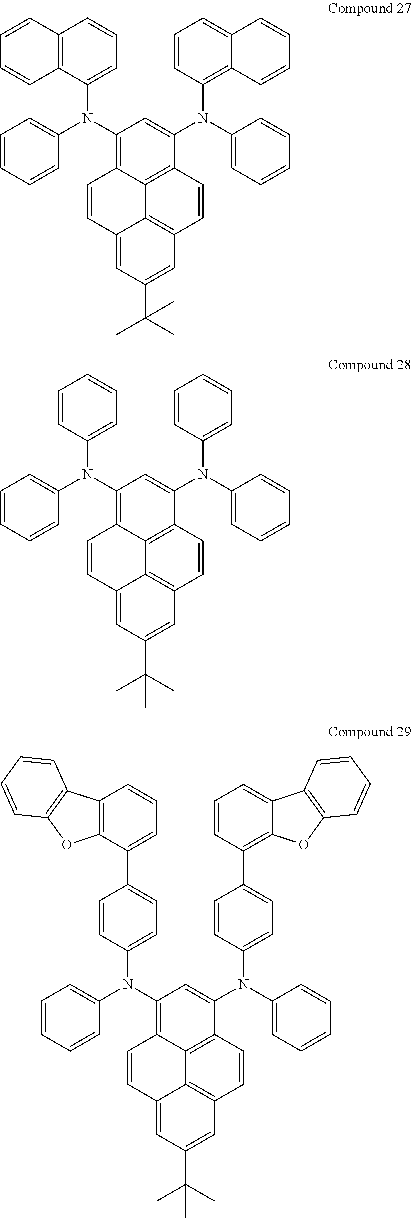 [SMILES: CC(C)(C)c1cc2ccc3c(N(c4ccccc4)c4cccc5ccccc45)cc(N(c4ccccc4)c4cccc5ccccc45)c4ccc(c1)c2c34 ; CC(C)(C)c1cc2ccc3c(N(c4ccccc4)c4ccccc4)cc(N(c4ccccc4)c4ccccc4)c4ccc(c1)c2c34 ; CC(C)(C)c1cc2ccc3c(N(c4ccccc4)c4ccc(-c5cccc6c5oc5ccccc56)cc4)cc(N(c4ccccc4)c4ccc(-c5cccc6c5oc5ccccc56)cc4)c4ccc(c1)c2c34]

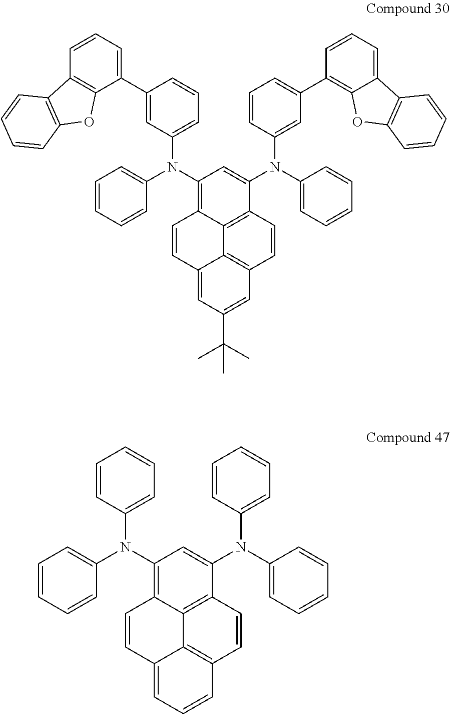 [SMILES: CC(C)(C)c1cc2ccc3c(N(c4ccccc4)c4cccc(-c5cccc6c5oc5ccccc56)c4)cc(N(c4ccccc4)c4cccc(-c5cccc6c5oc5ccccc56)c4)c4ccc(c1)c2c34 ; c1ccc(N(c2ccccc2)c2cc(N(c3ccccc3)c3ccccc3)c3ccc4cccc5ccc2c3c54)cc1]

### 4. Compounds Having Formula III

In some embodiments, the compounds described herein have Formula III

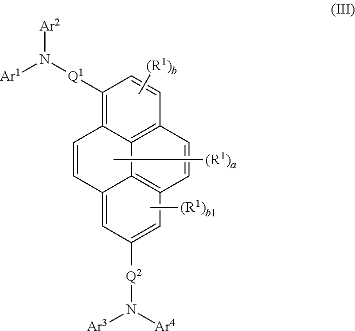

wherein:
Q¹ and Q² are the same or different and are selected from a single bond, hydrocarbon aryl and deuterated hydrocarbon aryl;
Ar¹-Ar⁴ are the same or different and are selected from the group consisting of hydrocarbon aryl, heteroaryl, substituted derivatives thereof, and deuterated analogs thereof, wherein Ar¹ and Ar² may be joined to form a carbazole group and Ar³ and Ar⁴ may be joined to form a carbazole group;
R¹ is the same or different at each occurrence and is selected from the group consisting of D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, and deuterated germyl;
a is an integer of 0-4; and
b and b1 are the same or different and are an integer of 0-2.

In some embodiments, the compounds having Formula III are useful as emissive materials. In some embodiments, the compounds are blue emissive materials. They can be used alone or as a dopant in a host material.

In some embodiments, compounds having Formula III have an unexpectedly narrow emission profile. In some embodiments, the emission profile has a width at half the maximum intensity (“FWHM”) that is less than 75 nm; in some embodiments, less than 60 nm; in some embodiments, less than 50 nm. This is advantageous for display devices for producing more saturated color.

In some embodiments, the compounds having Formula III have deep blue color. As used herein, the term “deep blue color” refers to a c.i.e. y-coordinate of less than 0.10, according to the C.I.E. chromaticity scale (Commission Internationale de L'Eclairage, 1931). In some embodiments, the compounds having Formula III have a photoluminescence y-coordinate of less than 0.10; in some embodiments, less than 0.090.

In some embodiments, devices including the compounds of Formula III have improved efficiencies. In some embodiments, the efficiency of a device including Formula III is greater than 4.5 cd/A at 1000 nits; in some embodiments, greater than 5.0 cd/A at 1000 nits.

In some embodiments, devices including the compounds of Formula III have increased lifetime. In some embodiments, devices including the compounds of Formula III have a T70 greater than 1000 hours at 50° C. As used herein, T70 refers to the time to reach 70% of initial luminance. In some embodiments, devices including the compounds of Formula III have a T70 greater than 1500 hours at 50° C.

In some embodiments, electroluminescent devices including the compounds of Formula III as emissive materials have deep blue color. In some embodiments, the x-coordinate is less than 0.15 and the y-coordinate is less than 0.10; in some embodiments, the y-coordinate is less than 0.090.

In some embodiments of Formula III, the compound is deuterated. In some embodiments, the compound is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments of Formula III, deuteration is present on the core pyrene group.

In some embodiments of Formula III, deuteration is present on one or more substituent groups.

In some embodiments of Formula III, deuteration is present on the core pyrene group and one or more substituent groups.

In some embodiments of Formula III, Q¹ is a single bond.

In some embodiments of Formula III, Q¹ is a hydrocarbon aryl or deuterated hydrocarbon aryl having no additional substituents.

In some embodiments of Formula III, Q¹ is a hydrocarbon aryl or deuterated hydrocarbon aryl having at least one substituent selected from the group consisting of F, CN, alkyl, silyl, deuterated alkyl, and deuterated silyl.

In some embodiments of Formula III, Q¹ has Formula c

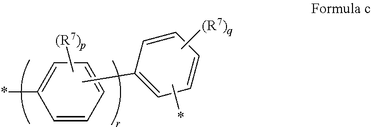

where p1 is an integer of 0-4, the asterisks indicate points of attachment and R⁷, p, and r are as in Formula a.

In some embodiments of Formula III, Q¹ has Formula d

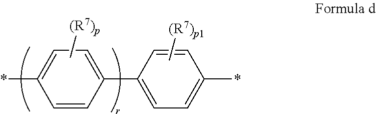

where the asterisks, R⁷, p, p1, and r are as in Formula c.

In some embodiments of Formula III, Q¹ is selected from phenyl, naphthyl, biphenyl, substituted derivatives thereof, and deuterated analogs thereof.

All of the above embodiments for Q¹ apply equally to Q².

In some embodiments of Formula III, Q¹=Q².

In some embodiments of Formula III, Q¹ ≠ Q².

In some embodiments of Formula III, at least one of Q¹ and Q² is a hydrocarbon aryl or substituted hydrocarbon aryl group.

In some embodiments of Formula III, at least one of Q¹ and Q² is a hydrocarbon aryl or substituted hydrocarbon aryl group and at least one of Q¹ and Q² is a single bond.

In some embodiments of Formula III, Q¹ is a hydrocarbon aryl or substituted hydrocarbon aryl group and Q² is a single bond.

In some embodiments of Formula III, at least one of Q¹ is a single bond and Q² is a hydrocarbon aryl or substituted hydrocarbon aryl group.

All of the embodiments for Ar¹, Ar², Ar³, Ar⁴, R¹, a, b, and b1 described above for Formula I, apply equally to Formula III.

In some embodiments of Formula III, the compounds have differently-substituted amino groups, where NAr¹Ar² ≠ NAr³Ar⁴.

In some embodiments of Formula III, the compounds have differently-substituted aryl-amino groups, where -Q¹NAr¹Ar² ≠-Q²NAr³Ar⁴.

In some embodiments of Formula III, the compound has Formula III-a

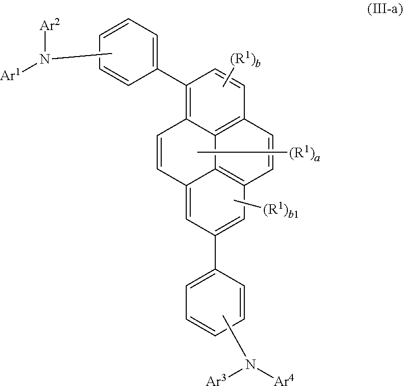

where Ar¹, Ar², Ar³, Ar⁴, R¹, a, b, and b1 are as described above for Formula III.

In some embodiments of Formula III, the compound has Formula III-b

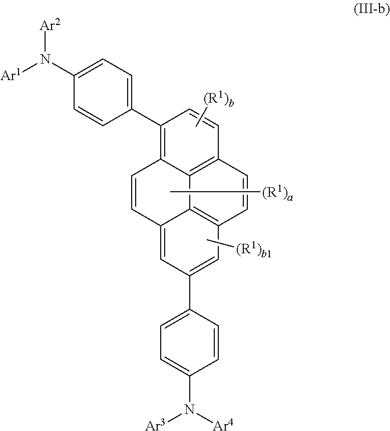

where Ar¹, Ar², Ar³, Ar⁴, R¹, a, b, and b1 are as described above for Formula III.

Any of the above embodiments of Formula III can be combined with one or more of the other embodiments, so long as they are not mutually exclusive. For example, the embodiment in which Ar¹ has a substituent that is an N,O-heteroaryl can be combined with the embodiment in which a=1 and at least one R¹ is a hydrocarbon aryl. The same is true for the other non-mutually-exclusive embodiments discussed above. The skilled person would understand which embodiments were mutually exclusive and would thus readily be able to determine the combinations of embodiments that are contemplated by the present application.

The compounds of Formula III can be made using any technique that will yield a C—C or C—N bond. A variety of such techniques are known, such as Suzuki, Stille, and metal-catalyzed C—N couplings as well as metal catalyzed and oxidative direct arylation.

As an illustrative example, Compound 32, shown below, may be prepared from the known 1,7-dibromopyrene via bis-Suzuki coupling with [3-(N-phenylanilino)phenyl]boronic acid as shown in Scheme 2.

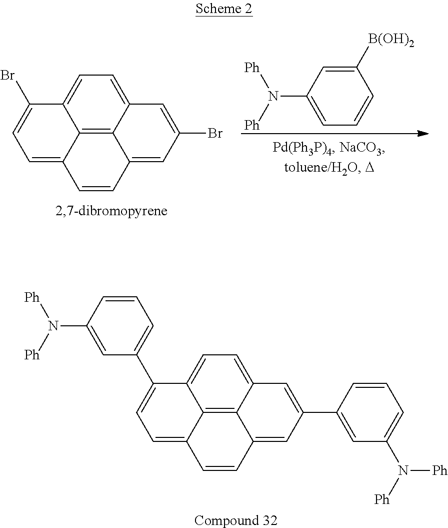

Compounds having Formula (III) and having differently-substituted aryl-amino groups (where -Q¹NAr¹Ar² ≠-Q²NAr³Ar⁴) may be prepared starting with Intermediate 5 as shown in Scheme 3.

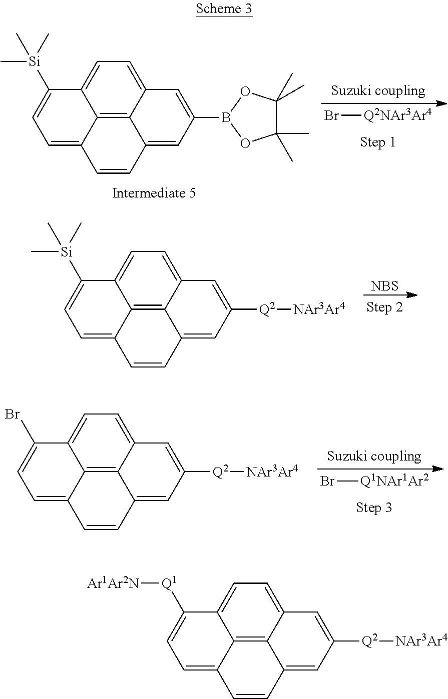

Deuterated compounds can be prepared in a similar manner using deuterated precursor materials or, more generally, by treating the non-deuterated compound with deuterated solvent, such as benzene-d6, in the presence of a Bronsted acid H/D exchange catalyst, such as trifluoromethanesulfonic acid, or a Lewis acid H/D exchange catalyst, such as aluminum trichloride or ethyl aluminum dichloride.

Exemplary preparations are given in the Examples.

Examples of compounds having Formula III include, but are not limited to, the compounds shown below.

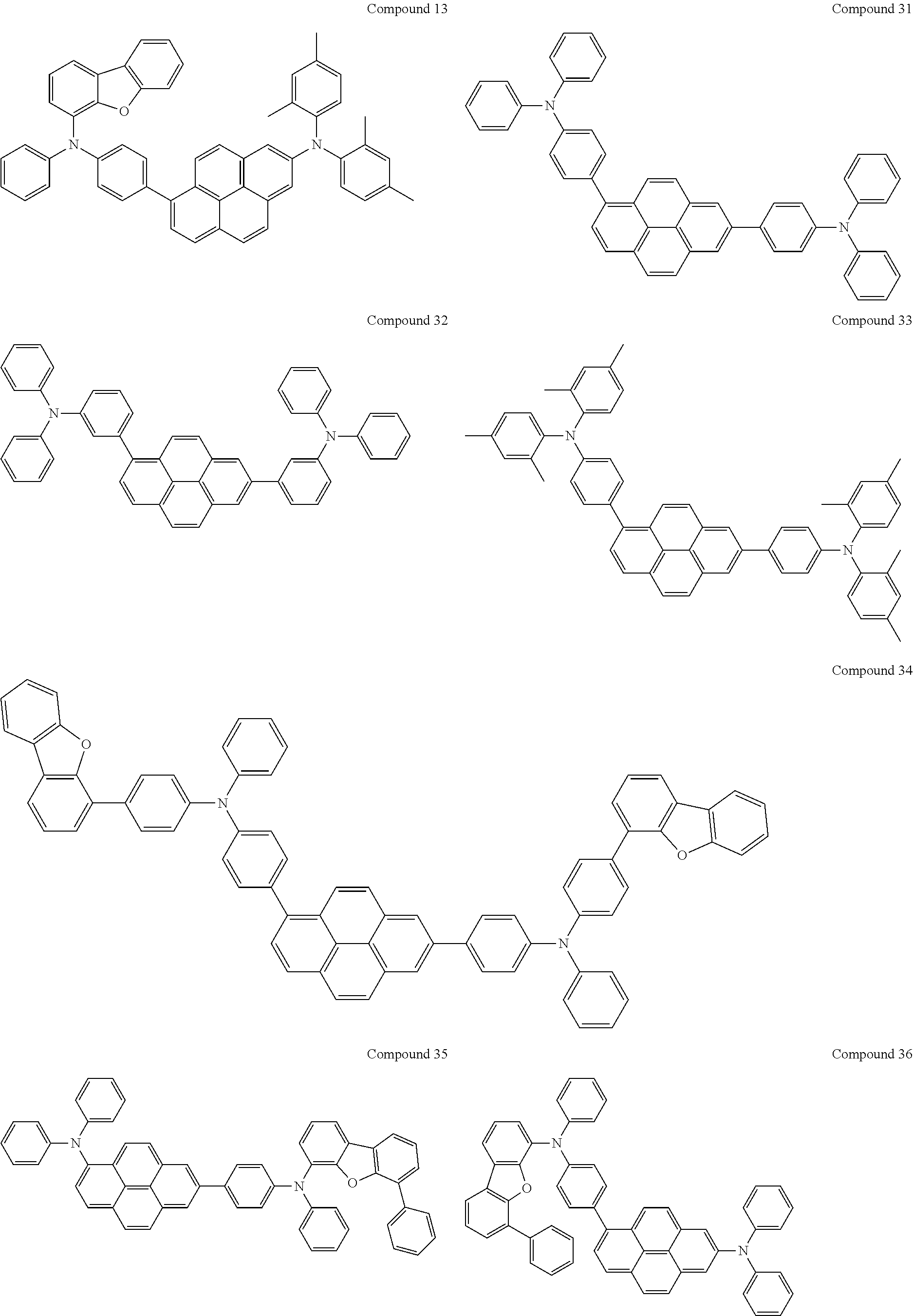

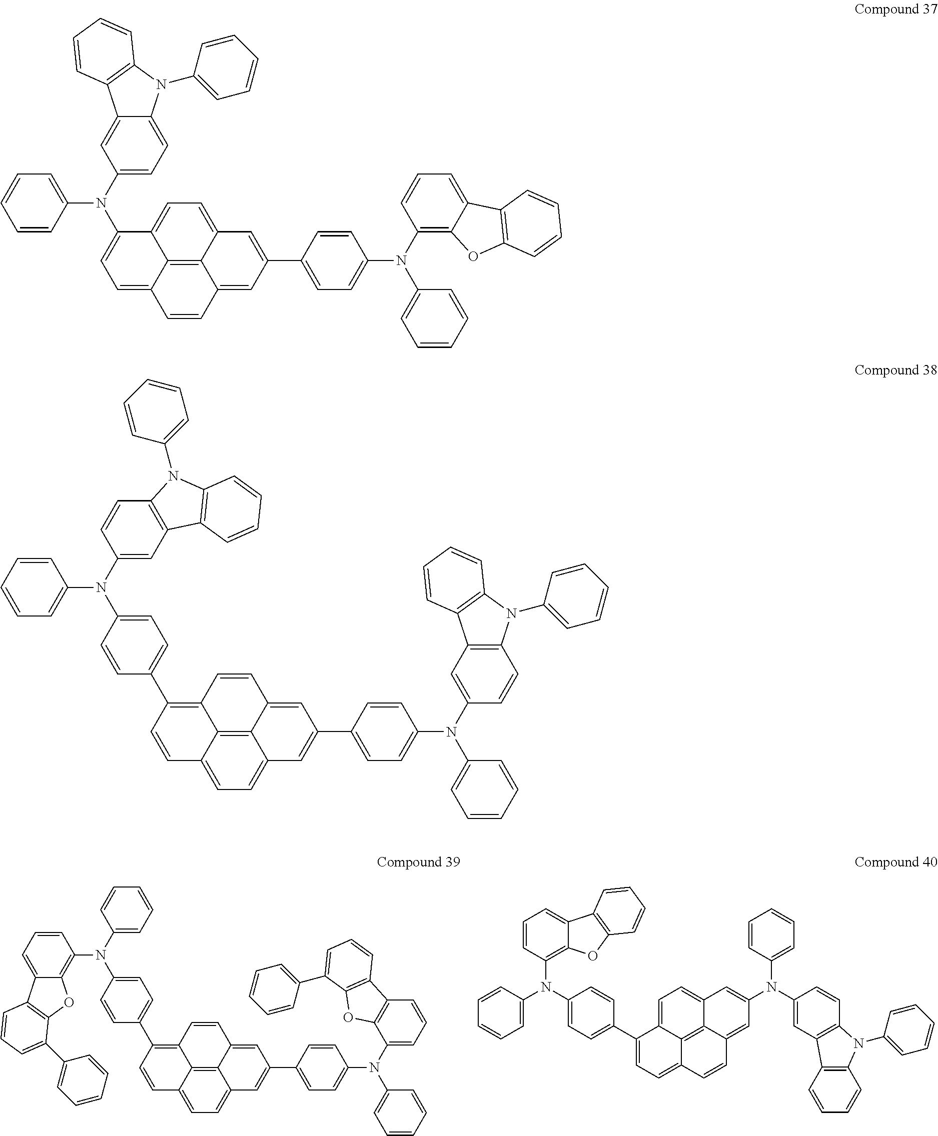

### 5. Compounds Having Formula IV

In some embodiments, the compounds described herein have Formula IV

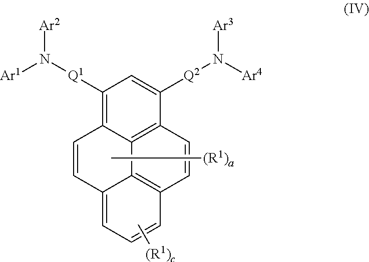

wherein:
Q¹ and Q² are the same or different and are selected from a single bond, hydrocarbon aryl and deuterated hydrocarbon aryl;
Ar¹-Ar⁴ are the same or different and are selected from the group consisting of hydrocarbon aryl, heteroaryl, substituted derivatives thereof, and deuterated analogs thereof, wherein Ar¹ and Ar² may be joined to form a carbazole group and Ar³ and Ar⁴ may be joined to form a carbazole group;
R¹ is the same or different at each occurrence and is selected from the group consisting of D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, and deuterated germyl;
a is an integer of 0-4; and
c is an integer of 0-3.

In some embodiments, the compounds having Formula IV are useful as emissive materials. In some embodiments, the compounds are blue emissive materials. They can be used alone or as a dopant in a host material.

In some embodiments, compounds having Formula IV have an unexpectedly narrow emission profile. In some embodiments, the emission profile has a width at half the maximum intensity (“FWHM”) that is less than 75 nm; in some embodiments, less than 60 nm; in some embodiments, less than 50 nm. This is advantageous for display devices for producing more saturated color.

In some embodiments, the compounds having Formula IV have deep blue color. As used herein, the term “deep blue color” refers to a c.i.e. y-coordinate of less than 0.10, according to the C.I.E. chromaticity scale (Commission Internationale de L'Eclairage, 1931). In some embodiments, the compounds having Formula IV have a photoluminescence y-coordinate of less than 0.10; in some embodiments, less than 0.090.

In some embodiments, devices including the compounds of Formula IV have improved efficiencies. In some embodiments, the efficiency of a device including Formula IV is greater than 4.5 cd/A at 1000 nits; in some embodiments, greater than 5.0 cd/A at 1000 nits.

In some embodiments, devices including the compounds of Formula IV have increased lifetime. In some embodiments, devices including the compounds of Formula IV have a T70 greater than 1000 hours at 50° C. As used herein, T70 refers to the time to reach 70% of initial luminance. In some embodiments, devices including the compounds of Formula IV have a T70 greater than 1500 hours at 50° C.

In some embodiments, electroluminescent devices including the compounds of Formula IV as emissive materials have deep blue color. In some embodiments, the x-coordinate is less than 0.15 and the y-coordinate is less than 0.10; in some embodiments, the y-coordinate is less than 0.090.

In some embodiments of Formula IV, the compound is deuterated. In some embodiments, the compound is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments of Formula IV, deuteration is present on the core pyrene group.

In some embodiments of Formula IV, deuteration is present on one or more substituent groups.

In some embodiments of Formula IV, deuteration is present on the core pyrene group and one or more substituent groups.

All of the embodiments for c described above for Formula II, apply equally to Formula IV.

All of the embodiments for Q¹, Q², Ar¹, Ar², Ar³, Ar⁴, R¹, and a described above for Formula III, apply equally to Formula IV.

In some embodiments of Formula IV, the compounds have differently-substituted amino groups.

In some embodiments of Formula IV, the compounds have differently-substituted aryl-amino groups.

In some embodiments of Formula IV, the compound has Formula IV-a

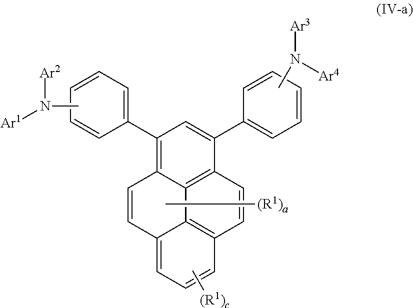

where Ar¹, Ar², Ar³, Ar⁴, R¹, a, b, and b1 are as described above for Formula IV.

In some embodiments of Formula IV, the compound has Formula IV-b

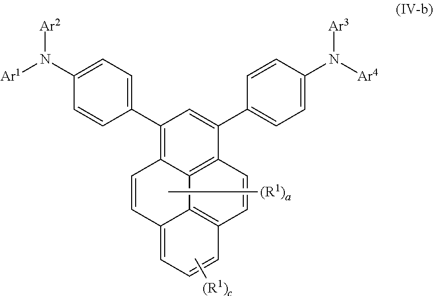

where Ar¹, Ar², Ar³, Ar⁴, R¹, a, b, and b1 are as described above for Formula IV.

Any of the above embodiments of Formula IV can be combined with one or more of the other embodiments, so long as they are not mutually exclusive. For example, the embodiment in which Ar¹ has a substituent that is an N,O-heteroaryl can be combined with the embodiment in which a=1 and at least one R¹ is a hydrocarbon aryl. The same is true for the other non-mutually-exclusive embodiments discussed above. The skilled person would understand which embodiments were mutually exclusive and would thus readily be able to determine the combinations of embodiments that are contemplated by the present application.

The compounds of Formula IV can be made using any technique that will yield a C—C or C—N bond. A variety of such techniques are known, such as Suzuki, Stille, and metal-catalyzed C—N couplings as well as metal catalyzed and oxidative direct arylation.

Deuterated compounds can be prepared in a similar manner using deuterated precursor materials or, more generally, by treating the non-deuterated compound with deuterated solvent, such as benzene-d6, in the presence of a Bronsted acid H/D exchange catalyst, such as trifluoromethanesulfonic acid, or a Lewis acid H/D exchange catalyst, such as aluminum trichloride or ethyl aluminum dichloride.

Exemplary preparations are given in the Examples.

Examples of compounds having Formula IV include, but are not limited to, the compounds shown below.

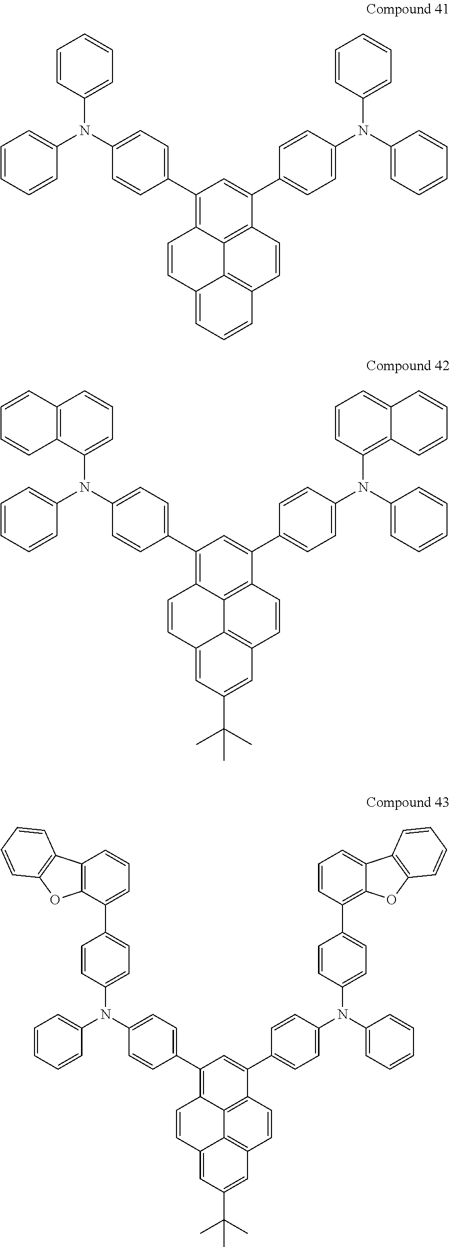 [SMILES: c1ccc(N(c2ccccc2)c2ccc(-c3cc(-c4ccc(N(c5ccccc5)c5ccccc5)cc4)c4ccc5cccc6ccc3c4c65)cc2)cc1 ; CC(C)(C)c1cc2ccc3c(-c4ccc(N(c5ccccc5)c5cccc6ccccc56)cc4)cc(-c4ccc(N(c5ccccc5)c5cccc6ccccc56)cc4)c4ccc(c1)c2c34 ; CC(C)(C)c1cc2ccc3c(-c4ccc(N(c5ccccc5)c5ccc(-c6cccc7c6oc6ccccc67)cc5)cc4)cc(-c4ccc(N(c5ccccc5)c5ccc(-c6cccc7c6oc6ccccc67)cc5)cc4)c4ccc(c1)c2c34]

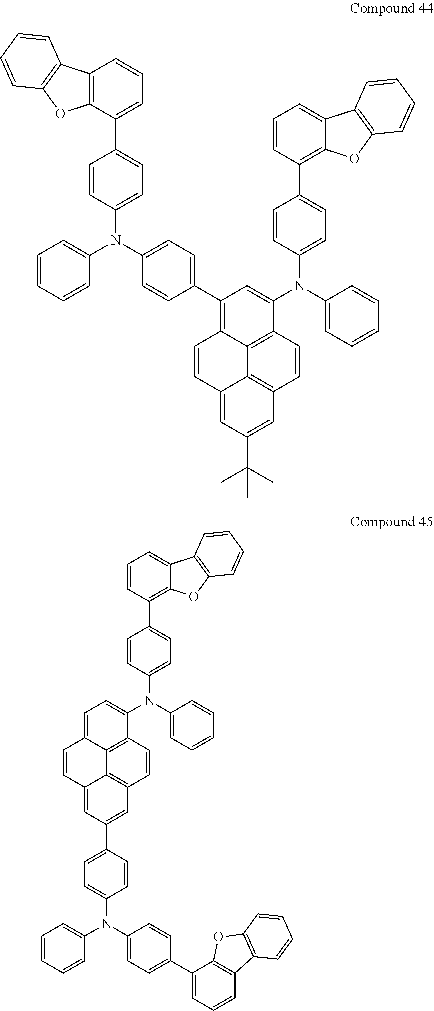

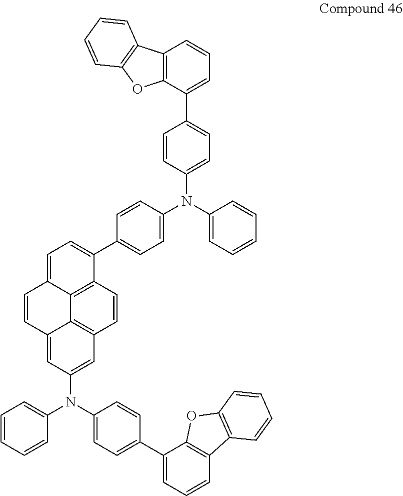 [SMILES: c1ccc(N(c2ccc(-c3ccc4ccc5cc(N(c6ccccc6)c6ccc(-c7cccc8c7oc7ccccc78)cc6)cc6ccc3c4c56)cc2)c2ccc(-c3cccc4c3oc3ccccc34)cc2)cc1]

### 6. Devices

Organic electronic devices that may benefit from having one or more layers comprising the compounds having Formulae I-IV described herein include, but are not limited to: (1) devices that convert electrical energy into radiation (e.g., a light-emitting diode, light emitting diode display, diode laser, or lighting panel; (2) devices that detect a signal using an electronic process (e.g., a photodetector, a photoconductive cell, a photoresistor, a photoswitch, a phototransistor, a phototube, an infrared (“IR”) detector, or a biosensors); (3) devices that convert radiation into electrical energy (e.g., a photovoltaic device or solar cell); (4) devices that convert light of one wavelength to light of a longer wavelength, (e.g., a down-converting phosphor device); (5) devices that include one or more electronic components that include one or more organic semiconductor layers (e.g., a transistor or diode); or any combination of devices in items (1) through (5).

In some embodiments, the device includes a photoactive layer having a compound of Formula I.

In some embodiments, the device includes a photoactive layer having a compound of Formula II.

In some embodiments, the device includes a photoactive layer having a compound of Formula III.

In some embodiments, the device includes a photoactive layer having a compound of Formula IV.

In some embodiments, the device includes an anode and a cathode with a photoactive layer therebetween, where the photoactive layer includes a compound having Formula I.

In some embodiments, the device includes an anode and a cathode with a photoactive layer therebetween, where the photoactive layer includes a compound having Formula II.

In some embodiments, the device includes an anode and a cathode with a photoactive layer therebetween, where the photoactive layer includes a compound having Formula III.

In some embodiments, the device includes an anode and a cathode with a photoactive layer therebetween, where the photoactive layer includes a compound having Formula IV.

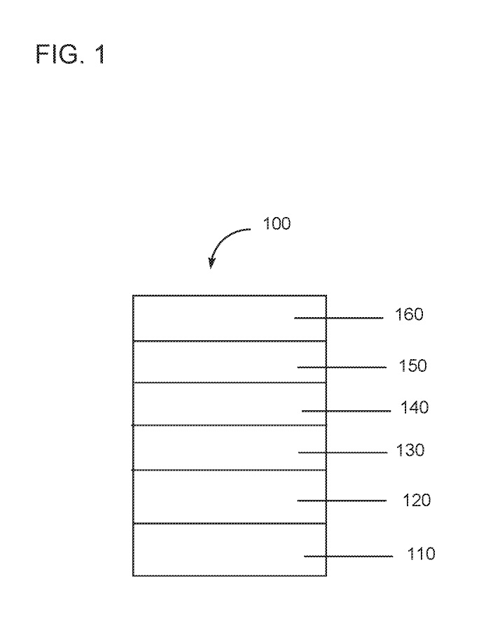

One illustration of an organic electronic device structure including a new compound as described herein is shown in FIG. 1. The device 100 has a first electrical contact layer, an anode layer 110 and a second electrical contact layer, a cathode layer 160, and a photoactive layer 140 between them. Adjacent to the anode is a hole injection layer 120. Adjacent to the hole injection layer is a hole transport layer 130, comprising hole transport material. Adjacent to the cathode may be an electron transport layer 150, comprising an electron transport material. As an option, devices may use one or more additional hole injection or hole transport layers (not shown) next to the anode 110 and/or one or more additional electron injection or electron transport layers (not shown) next to the cathode 160. As a further option, devices may have an anti-quenching layer (not shown) between the photoactive layer 140 and the electron transport layer 150.

Layers 120 through 150, and any additional layers between them, are individually and collectively referred to as the active layers.

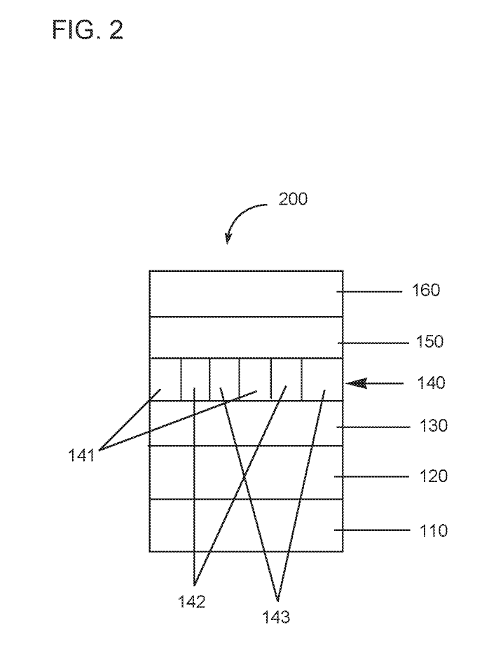

In some embodiments, the photoactive layer is pixellated, as shown in FIG. 2. In device 200, layer 140 is divided into pixel or subpixel units 141, 142, and 143 which are repeated over the layer. Each of the pixel or subpixel units represents a different color. In some embodiments, the subpixel units are for red, green, and blue. Although three subpixel units are shown in the figure, two or more than three may be used.

The different layers will be discussed further herein with reference to FIG. 1. However, the discussion applies to FIG. 2 and other configurations as well.

In some embodiments, the different layers have the following range of thicknesses: anode 110, 500-5000 Å, in some embodiments, 1000-2000 Å; hole injection layer 120, 50-2000 Å, in some embodiments, 200-1000 Å; hole transport layer 130, 50-2000 Å, in some embodiments, 200-1000 Å; photoactive layer 140, 10-2000 Å, in some embodiments, 100-1000 Å; electron transport layer 150, 50-2000 Å, in some embodiments, 100-1000 Å; cathode 160, 200-10000 Å, in some embodiments, 300-5000 Å. The location of the electron-hole recombination zone in the device, and thus the emission spectrum of the device, can be affected by the relative thickness of each layer. The desired ratio of layer thicknesses will depend on the exact nature of the materials used.

In some embodiments, the compounds having Formulae I-IV are useful as the emissive material in photoactive layer 140, having blue emission color. They can be used alone or as a dopant in a host material.

a. Photoactive Layer

In some embodiments, the photoactive layer includes a host material and a compound having Formulae I-IV as a dopant. In some embodiments, a second host material is present.

In some embodiments, the photoactive layer includes only a host material and a compound having Formulae I-IV as a dopant. In some embodiments, minor amounts of other materials, are present so long as they do not significantly change the function of the layer.

In some embodiments, the photoactive layer includes only a first host material, a second host material, and a compound having Formulae I-IV as a dopant. In some embodiments, minor amounts of other materials, are present so long as they do not significantly change the function of the layer.

The weight ratio of dopant to total host material is in the range of 2:98 to 50:50; in some embodiments, 3:97 to 30:70; in some embodiments, 5:95 to 20:80.

In some embodiments, the host material is selected from the group consisting of chrysenes, phenanthrenes, triphenylenes, phenanthrolines, triazines, naphthalenes, anthracenes, quinolines, isoquinolines, quinoxalines, phenylpyridines, carbazoles, indolocarbazoles, indoloindoles, furans, benzofurans, dibenzofurans, benzodifurans, naphthofurans, naphthodifurans, metal quinolinate complexes, substituted derivatives thereof, deuterated analogs thereof, and combinations thereof.

In some embodiments, the host is selected from the group consisting of triphenylenes, anthracenes, indolocarbazoles, inoloindoles, furans, benzofurans, dibenzofurans, benzodifurans, naphthodifurans, substituted derivatives thereof, deuterated analogs thereof, and combinations thereof.

In some embodiments, the host material is a 9,10-diaryl anthracene compound or deuterated analog thereof.

In some embodiments, the host material is a chrysene derivative having one or two diarylamino substituents, or a deuterated analog thereof.

In some embodiments, the host material is a naphthodifuran, substituted derivative thereof, or a deuterated analog thereof.

Any of the compounds of Formulae I-IV represented by the embodiments, specific embodiments, specific examples, and combination of embodiments discussed above can be used in the photoactive layer.

b. Other Device Layers

The other layers in the device can be made of any materials which are known to be useful in such layers.

The anode 110 is an electrode that is particularly efficient for injecting positive charge carriers. It can be made of, for example materials containing a metal, mixed metal, alloy, metal oxide or mixed-metal oxide, or it can be a conducting polymer, and mixtures thereof. Suitable metals include the Group 11 metals, the metals in Groups 4, 5, and 6, and the Group 8-10 transition metals. If the anode is to be light-transmitting, mixed-metal oxides of Groups 12, 13 and 14 metals, such as indium-tin-oxide, are generally used. The anode may also be made of an organic material such as polyaniline as described in “Flexible light-emitting diodes made from soluble conducting polymer,” Nature vol. 357, pp 477 479 (11 Jun. 1992). At least one of the anode and cathode should be at least partially transparent to allow the generated light to be observed.

The hole injection layer 120 includes hole injection material and may have one or more functions in an organic electronic device, including but not limited to, planarization of the underlying layer, charge transport and/or charge injection properties, scavenging of impurities such as oxygen or metal ions, and other aspects to facilitate or to improve the performance of the organic electronic device. The hole injection layer can be formed with polymeric materials, such as polyaniline (PANI) or polyethylenedioxythiophene (PEDOT), which are often doped with protonic acids. The protonic acids can be, for example, poly(styrenesulfonic acid), poly(2-acrylamido-2-methyl-1-propanesulfonic acid), and the like.

The hole injection layer can include charge transfer compounds, and the like, such as copper phthalocyanine and the tetrathiafulvalene-tetracyanoquinodimethane system (TTF-TCNQ).

In some embodiments, the hole injection layer includes at least one electrically conductive polymer and at least one fluorinated acid polymer.

In some embodiments, the hole injection layer is made from an aqueous dispersion of an electrically conducting polymer doped with a colloid-forming polymeric acid. Such materials have been described in, for example, published U.S. patent applications US 2004/0102577, US 2004/0127637, US 2005/0205860, and published PCT application WO 2009/018009.

Examples of hole transport materials for layer 130 have been summarized for example, in Kirk-Othmer Encyclopedia of Chemical Technology, Fourth Edition, Vol. 18, p. 837-860, 1996, by Y. Wang. Both hole transporting molecules and polymers can be used. Commonly used hole transporting molecules are: N,N′-diphenyl-N,N′-bis(3-methylphenyl)-[1,1′-biphenyl]-4,4′-diamine (TPD), 1,1-bis[(di-4-tolylamino) phenyl]cyclohexane (TAPC), N,N′-bis(4-methylphenyl)-N,N-bis(4-ethylphenyl)-[1,1′-(3,3′-dimethyl)biphenyl]-4,4′-diamine (ETPD), tetrakis-(3-methylphenyl)-N,N,N′,N′-2,5-phenylenediamine (PDA), a-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl] pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), N,N′-bis(naphthalen-1-yl)-N,N′-bis-(phenyl)benzidine (□-NPB), and porphyrinic compounds, such as copper phthalocyanine. In some embodiments, the hole transport layer includes a hole transport polymer. In some embodiments, the hole transport polymer is a distyrylaryl compound. In some embodiments, the aryl group has two or more fused aromatic rings. In some embodiments, the aryl group is an acene. The term “acene” as used herein refers to a hydrocarbon parent component that contains two or more ortho-fused benzene rings in a straight linear arrangement. Other commonly used hole transporting polymers are polyvinylcarbazole, (phenylmethyl)-polysilane, and polyaniline. It is also possible to obtain hole transporting polymers by doping hole transporting molecules such as those mentioned above into polymers such as polystyrene and polycarbonate. In some cases, triarylamine polymers are used, especially triarylamine-fluorene copolymers. In some cases, the polymers and copolymers are crosslinkable.

In some embodiments, the hole transport layer further includes a p-dopant. In some embodiments, the hole transport layer is doped with a p-dopant. Examples of p-dopants include, but are not limited to, tetrafluorotetracyanoquinodimethane (F4-TCNQ) and perylene-3,4,9,10-tetracarboxylic-3,4,9,10-dianhydride (PTCDA).

In some embodiments, more than one hole transport layer is present (not shown).

Examples of electron transport materials which can be used for layer 150 include, but are not limited to, metal chelated oxinoid compounds, including metal quinolate derivatives such as tris(8-hydroxyquinolato)aluminum (AlQ), bis(2-methyl-8-quinolinolato)(p-phenylphenolato) aluminum (BAlq), tetrakis-(8-hydroxyquinolato)hafnium (HfQ) and tetrakis-(8-hydroxyquinolato)zirconium (ZrQ); and azole compounds such as 2-(4-biphenylyl)-5-(4-t-butylphenyl)-1,3,4-oxadiazole (PBD), 3-(4-biphenylyl)-4-phenyl-5-(4-t-butylphenyl)-1,2,4-triazole (TAZ), and 1,3,5-tri(phenyl-2-benzimidazole)benzene (TP61); quinoxaline derivatives such as 2,3-bis(4-fluorophenyl)quinoxaline; fluoranthene derivatives, such as 3-(4-(4-methylstyryl)phenyl-p-tolylamino)fluoranthene; phenanthrolines such as 4,7-diphenyl-1,10-phenanthroline (DPA) and 2,9-dimethyl-4,7-diphenyl-1,10-phenanthroline (DDPA); and mixtures thereof. In some embodiments, the electron transport layer further includes an n-dopant. N-dopant materials are well known. The n-dopants include, but are not limited to, Group 1 and 2 metals; Group 1 and 2 metal salts, such as LiF, CsF, and Cs₂CO₃; Group 1 and 2 metal organic compounds, such as Li quinolate; and molecular n-dopants, such as leuco dyes, metal complexes, such as W2(hpp)₄ where hpp=1,3,4,6,7,8-hexahydro-2H-pyrimido-[1,2-a]-pyrimidine and cobaltocene, tetrathianaphthacene, bis(ethylenedithio)tetrathiafulvalene, heterocyclic radicals or diradicals, and the dimers, oligomers, polymers, dispiro compounds and polycycles of heterocyclic radical or diradicals.

In some embodiments, an anti-quenching layer may be present between the photoactive layer and the electron transport layer to prevent quenching of blue luminance by the electron transport layer. To prevent energy transfer quenching, the singlet energy of the anti-quenching material has to be higher than the singlet energy of the blue emitter. To prevent electron transfer quenching, the LUMO level of the anti-quenching material has to be shallow enough (with respect to the vacuum level) such that electron transfer between the emitter exciton and the anti-quenching material is endothermic. Furthermore, the HOMO level of the anti-quenching material has to be deep enough (with respect to the vacuum level) such that electron transfer between the emitter exciton and the anti-quenching material is endothermic. In general, anti-quenching material is a large band-gap material with high singlet and triplet energies.

The cathode 160, is an electrode that is particularly efficient for injecting electrons or negative charge carriers. The cathode can be any metal or nonmetal having a lower work function than the anode. Materials for the cathode can be selected from alkali metals of Group 1 (e.g., Li, Cs), the Group 2 (alkaline earth) metals, the Group 12 metals, including the rare earth elements and lanthanides, and the actinides. Materials such as aluminum, indium, calcium, barium, samarium and magnesium, as well as combinations, can be used.

Alkali metal-containing inorganic compounds, such as LiF, CsF, Cs₂O and Li₂O, or Li-containing organometallic compounds can also be deposited between the organic layer 150 and the cathode layer 160 to lower the operating voltage. This layer, not shown, may be referred to as an electron injection layer.

It is known to have other layers in organic electronic devices. For example, there can be a layer (not shown) between the anode 110 and hole injection layer 120 to control the amount of positive charge injected and/or to provide band-gap matching of the layers, or to function as a protective layer. Layers that are known in the art can be used, such as copper phthalocyanine, silicon oxy-nitride, fluorocarbons, silanes, or an ultra-thin layer of a metal, such as Pt. Alternatively, some or all of anode layer 110, active layers 120, 130, 140, and 150, or cathode layer 160, can be surface-treated to increase charge carrier transport efficiency. The choice of materials for each of the component layers is preferably determined by balancing the positive and negative charges in the emitter layer to provide a device with high electroluminescence efficiency.

It is understood that each functional layer can be made up of more than one layer.

c. Device Fabrication

The device layers can be formed by any deposition technique, or combinations of techniques, including vapor deposition, liquid deposition, and thermal transfer.

In some embodiments, the device is fabricated by vapor deposition of all of the layers.

In some embodiments, the device is fabricated by liquid deposition of the hole injection layer, the hole transport layer, and the photoactive layer, and by vapor deposition of the anode, the electron transport layer, an electron injection layer and the cathode.

The hole injection layer can be deposited from any liquid medium in which it is dissolved or dispersed and from which it will form a film. In some embodiments, the liquid medium includes only one or more organic solvents. In some embodiments, minor amounts of other materials are present, so long as they do not substantially affect the liquid medium.

In some embodiments, the liquid medium includes only water or includes only water and an organic solvent. In some embodiments, minor amounts of other materials are present, so long as they do not substantially affect the liquid medium.

The hole injection material is present in the liquid medium in an amount from 0.5 to 10 percent by weight.

In some embodiments, the hole injection layer is formed by any continuous or discontinuous liquid deposition technique. In some embodiments, the hole injection layer is applied by spin coating. In some embodiments, the hole injection layer is applied by ink jet printing. In some embodiments, the hole injection layer is applied by continuous nozzle printing. In some embodiments, the hole injection layer is applied by slot-die coating. After liquid deposition, the liquid medium can be removed in air, in an inert atmosphere, or by vacuum, at room temperature or with heating.

In some embodiments, the hole transport layer is formed by liquid deposition of hole transport material and any additional additives in a liquid medium. The liquid medium is one in which the materials of the hole transport layer are dissolved or dispersed and from which a film will be formed. In some embodiments, the liquid medium includes one or more organic solvents. In some embodiments, the organic solvent is an aromatic solvent. In some embodiments, the organic liquid is selected from chloroform, dichloromethane, chlorobenzene, dichlorobenzene, toluene, xylene, mesitylene, anisole, N-methyl-2-pyrrolidone, tetralin, 1-methoxynaphthalene, cyclohexylbenzene, and mixtures thereof. The hole transport material can be present in the liquid medium in a concentration of 0.2 to 5 percent (w/v); in some embodiments, 0.4 to 3 percent (w/v). The hole transport layer can be applied by any continuous or discontinuous liquid deposition technique. In some embodiments, the hole transport layer is applied by spin coating. In some embodiments, the hole transport layer is applied by ink jet printing. In some embodiments, the hole transport layer is applied by continuous nozzle printing. In some embodiments, the hole transport layer is applied by slot-die coating. After liquid deposition, the liquid medium can be removed in air, in an inert atmosphere, or by vacuum, at room temperature or with heating.

In some embodiments, the photoactive layer is formed by vapor deposition. Such techniques are well known in the art.

In some embodiments, the photoactive layer is formed by liquid deposition of the photoactive material and one or more host materials in a liquid medium. The liquid medium is one in which the materials of the photoactive layer are dissolved or dispersed and from which they will form a film. In some embodiments, the liquid medium includes one or more organic solvents. In some embodiments, minor amounts of additional materials are present so long as they do not substantially affect the function of the photoactive layer.

Suitable classes of solvents include, but are not limited to, aliphatic hydrocarbons (such as decane, hexadecane, and decalin), halogenated hydrocarbons (such as methylene chloride, chloroform, chlorobenzene, benzotrifluoride, and perfluoroheptane), aromatic hydrocarbons (such as non-substituted and alkyl- and alkoxy-substituted benzenes, toluenes and xylenes), aromatic ethers (such as anisole, dibenzyl ether, and fluorinated derivatives), heteroaromatics (such as pyridine) polar solvents (such as tetrahydropyran, dimethylacetamide, N-methyl pyrrolidone, and nitriles such as acetonitrile), esters (such as ethylacetate, propylene carbonate, methyl benzoate, and phosphate esters such as tributylphosphate), alcohols and glycols (such as isopropanol and ethylene glycol), glycol ethers and derivatives (such as propylene glycol methyl ether and propylene glycol methyl ether acetate), and ketones (such as cyclopentanone and diisobutyl ketone).

The photoactive material can be present in the liquid medium in a concentration of 0.2 to 5 percent by weight; in some embodiments, 0.2 to 2 percent by weight. Other weight percentages of photoactive material may be used depending upon the liquid medium. The photoactive layer can be applied by any continuous or discontinuous liquid deposition technique. In some embodiments, the photoactive layer is applied by spin coating. In some embodiments, the photoactive layer is applied by ink jet printing. In some embodiments, the photoactive layer is applied by continuous nozzle printing. In some embodiments, the photoactive layer is applied by slot-die coating. After liquid deposition, the liquid medium can be removed in air, in an inert atmosphere, or by vacuum, at room temperature or with heating.

The electron transport layer can be deposited by any vapor deposition method. In some embodiments, it is deposited by thermal evaporation under vacuum.

The electron injection layer can be deposited by any vapor deposition method. In some embodiments, it is deposited by thermal evaporation under vacuum.

The cathode can be deposited by any vapor deposition method. In some embodiments, it is deposited by thermal evaporation under vacuum.

### EXAMPLES

The concepts described herein will be further described in the following examples, which do not limit the scope of the invention described in the claims.

### Synthesis Example 1

This example illustrates the preparation of a compound having Formula I, Compound 2.

a. Synthesis of 1-bromo-7-(4,4,5,5,-tetramethyl-1,3,2-dioxaborylan-2-yl)pyrene (Intermediate 1).

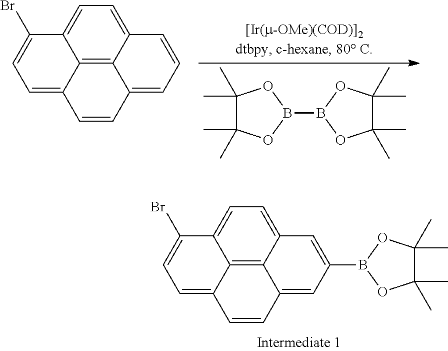

A solution of 419 mg of the iridium pre-catalyst [Ir(μ-OMe)(COD)]₂, 339 mg of 4,4′-di-tert-butyl-2,2′-dipyridyl (dtbpy), and 700 mg of bis(pinacolato)diboron in 33 mL of cyclohexane was stirred for 13 minutes whereupon it was added to a mixture of 17.77 g of 1-bromopyrene and 16.95 g of bis(pinacolato)diboron in 102 mL cyclohexane. The reaction was heated at bath temperature (T_{b}) 65° C. overnight. Upon cooling, the crude reaction mixture was concentrated, and purified by medium pressure liquid chromatography (MPLC) on silica gel eluting with 7:3 dichloromethane:hexane. The purest fractions were combined and concentrated by rotary evaporation to give Intermediate 1 as a white solid (4.2 g, 16% yield).

b. Synthesis of 1,7-dibromopyrene (Intermediate 2).

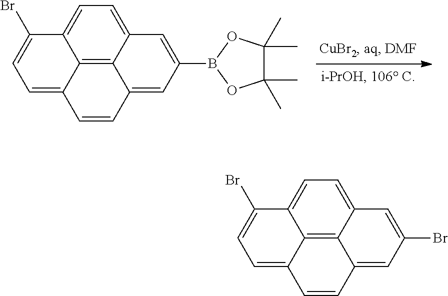 [SMILES: CC1(C)OB(c2cc3ccc4ccc(Br)c5ccc(c2)c3c45)OC1(C)C ; Brc1cc2ccc3ccc(Br)c4ccc(c1)c2c34]

A mixture of 2.92 g of Intermediate 1 in 28 mL isopropanol and 28 mL DMF was treated with 3.6 g of CuBr₂ in 28 mL of water. The reaction was heated at T_{b} 106° C. under nitrogen. After 5.75 hours, an additional 210 mg of CuBr₂ was added. After 7.25 h the reaction was complete. The reaction was cooled to room temperature, 300 mL of water was added, and the mixture filtered. The solid was washed with 300 mL water, then 280 mL methanol, and finally dried under vacuum at 62° C. for 30 minutes to give Intermediate 2 as an off-white solid (4.2 g, quantitative yield).

c. Compound 2

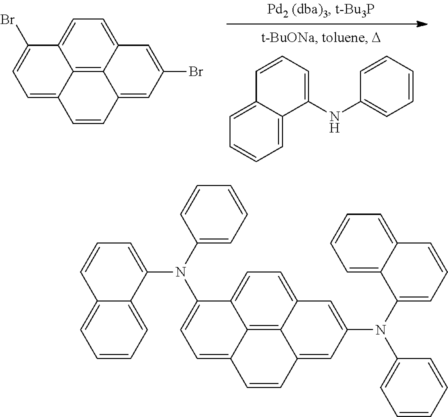

To 2.1 g of Intermediate 2 in 100 mL toluene under nitrogen atmosphere was added 36 mg of P(t-Bu)₃ and 323 mg of Pd₂(dba)₃. Next, 2.17 g of N-phenyl-1-naphthylamine and 951 mg of sodium tert-butoxide were added. The reaction was heated at reflux for 3.25 hours, cooled to room temperature and filtered through a plug of Celite. Water was added to the filtrate and the mixture was extracted with dichloromethane. The combined organic layers were dried over sodium sulfate, filtered, and concentrated by rotary evaporation to give a dark amber oil. The crude material was dissolved in 5:3 hexanes:dichloromethane and purified by MPLC on silica gel eluting with 90:10 to 65:35 hexanes:dichloromethane. The purest fractions (analyzed by UPLC) were combined and concentrated by rotary evaporation to provide Compound 2 (700 mg, 99.74% pure) as a yellow solid. Final purification prior to device preparation was accomplished by vacuum sublimation.

### Synthesis Example 2

This example illustrates the preparation of a compound having Formula I, Compound 5.

a. Synthesis of 2,2′,4,4′-tetramethyldiphenylamine (Intermediate 3).

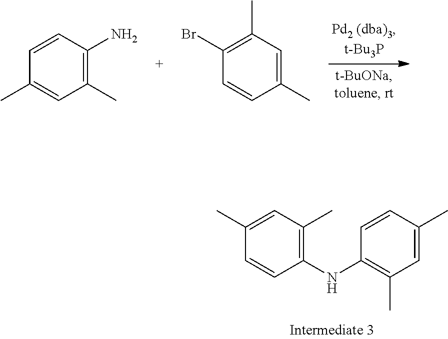

To 6.0 g of 2,4-dimethylaniline, 8.7 g of 1-bromo-2,4-dimethylbenzene, 916 mg of Pd₂(dba)₃, and 404 mg of P(t-Bu)₃ in 400 mL toluene was added 5.29 g of sodium tert-butoxide. The reaction was stirred at room temperature. After 40 hours, water and brine were added. The toluene layer was separated and the aqueous layer was extracted with dichloromethane. The organic layers were combined, dried over sodium sulfate, and concentrated by rotary evaporation. The crude material was purified by MPLC on silica gel eluting with 95:5 to 80:20 hexanes:dichloromethane, combining the purest fractions to give, after concentration by rotary evaporation, Intermediate 3 (8.7 g, 77% yield) as a colorless liquid.

b. Compound 5

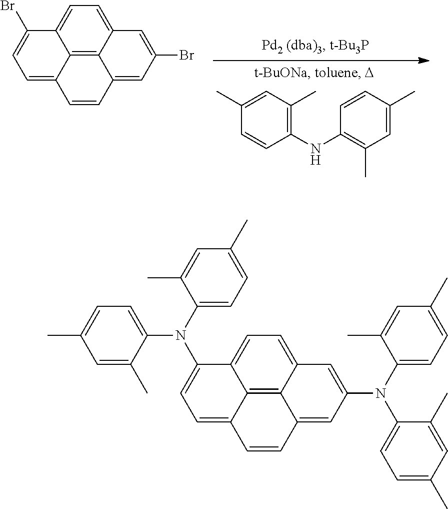

To a mixture of 300 mg of Intermediate 2 in 14 mL toluene under nitrogen atmosphere was added 5 mg of P(t-Bu)₃ and 46 mg of Pd₂(dba)₃. Next, 376 mg of Intermediate 3 and 161 mg of sodium tert-butoxide were added. The reaction was heated at reflux for 2 hours. After the reaction was cooled, water was added and the contents were extracted with dichloromethane. The combined extracts were dried over sodium sulfate, filtered, and concentrated by rotary evaporation. The crude material was purified by MPLC on silica gel eluting with 95:5 to 75:25 hexanes:dichloromethane. Fractions containing the desired compound were combined, concentrated by rotary evaporation, then dissolved in hexanes/ethyl acetate. After slow evaporation of the solvents, the resulting solid was triturated with hot dichloromethane to provide Compound 5 (52 mg, 10% yield, 99.62% pure) as a gold-orange solid.

### Synthesis Example 3

This example illustrates the preparation of a compound having Formula I, Compound 24.

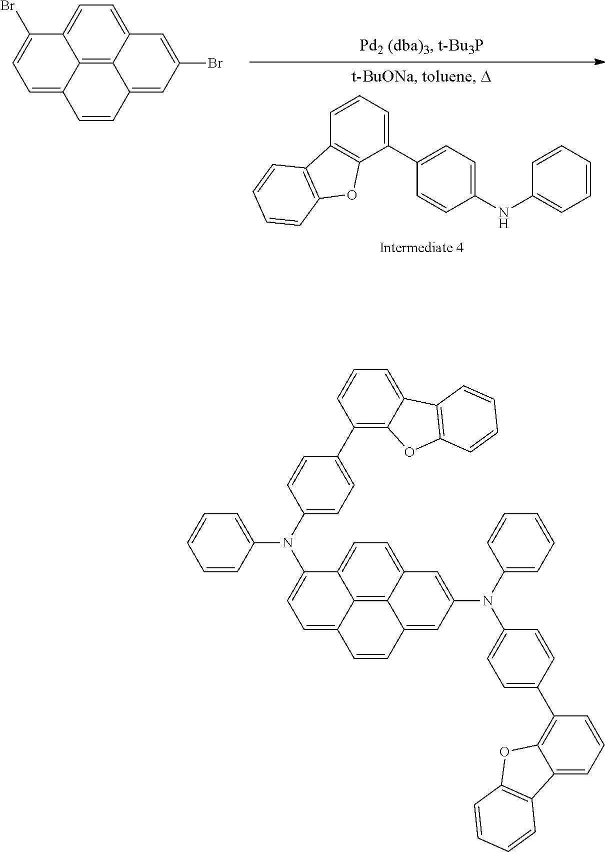 [SMILES: Brc1cc2ccc3ccc(Br)c4ccc(c1)c2c34 ; c1ccc(Nc2ccc(-c3cccc4c3oc3ccccc34)cc2)cc1 ; c1ccc(N(c2ccc(-c3cccc4c3oc3ccccc34)cc2)c2cc3ccc4ccc(N(c5ccccc5)c5ccc(-c6cccc7c6oc6ccccc67)cc5)c5ccc(c2)c3c45)cc1]

### Compound 24

To 2.82 g of Intermediate 2 in 100 mL toluene was added a solution of 46.5 mg of P(t-Bu)₃ and 421 mg of Pd₂(dba)₃ in 12 mL toluene. Next, 5.2 g of Intermediate 4 (prepared in the manner reported on page 36 of US20120181521-A1) in 5 mL toluene and 1.49 g of sodium tert-butoxide in 5 mL toluene were added. The reaction was heated at T_{b}=92° C. After 3 hours, the reaction was cooled, water was added, and the mixture was extracted with dichloromethane. The combined extracts were dried over sodium sulfate, filtered through Celite, and concentrated by rotary evaporation to give a brown oil. The crude material was purified by MPLC on silica gel eluting with 95:5 to 60:40 hexanes:dichloromethane. The purest fractions were combined and triturated with 1:1 acetonitrile:dichloromethane to give 2.25 g of the product as a yellow solid.

The sample was re-purified by MPLC on silica gel eluting with 90:10 to 60:40 hexanes:dichloromethane to give the product (785 mg). The less pure fractions were combined with the filtrate from the from the 1:1 acetonitrile:dichloromethane trituration. Both lots were recrystallized from toluene/methanol and combined to give Compound 24 (1.32 g, 99.98% pure) as a yellow solid. Final purification prior to device preparation was accomplished by vacuum sublimation.

### Synthesis Example 4

This example illustrates the preparation of an intermediate which can be used to prepare compounds having Formula I with differently substituted amino groups, intermediate trimethyl[7-(4,4,5,5-tetramethyl-1,3,2-dioxaborolan-2-yl)pyren-1-yl]silane, Intermediate 5.

a. Synthesis of trimethyl(pyren-1-yl)silane.

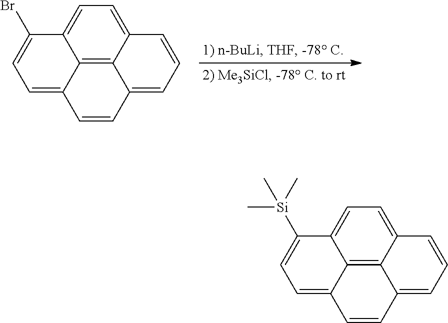 [SMILES: Brc1ccc2ccc3cccc4ccc1c2c34 ; C[Si](C)(C)c1ccc2ccc3cccc4ccc1c2c34]

To a chilled solution (T_{b}=−78° C.) of 60 g of 1-bromopyrene in 1,300 mL of dry tetrahydrofuran was added 132 mL of 2.43M n-butyl lithium) in THF. After stirring for 1 h, 41.5 g of chlorotrimethylsilane was added to the −78° C. reaction mixture which was then allowed to warm to room temperature. Analysis of a quenched reaction aliquot indicated that the reaction was complete. The mixture was then cooled back to 0° C., quenched with saturated aqueous ammonium chloride, and extracted with petroleum ether. The combined organic layer was dried over sodium sulfate, filtered and concentrated by rotary evaporation. The crude product was combined with the similarly obtained product from a 10 g scale reaction and was purified by silica gel column chromatography eluting with petroleum ether. The pure fractions thus obtained were concentrated to dryness and washed three times successively with ethanol to give trimethyl(pyren-1-yl)silane (40 g, 59% combined yield).

b. Synthesis of Intermediate 5

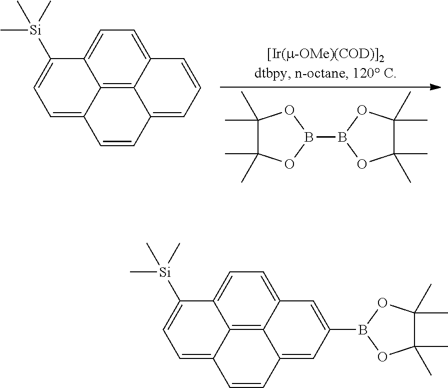

A mixture of 20 g of trimethyl(pyren-1-yl)silane and 33.3 g of bis(pinacolato)diboron in 670 mL of n-octane was purged with argon for 15 min. The iridium pre-catalyst [Ir(μ-OMe)(COD)]₂, (483 mg, 0.73 mmol) and 391 mg of 4,4′-di-tert-butyl-2,2′-dipyridyl (dtbpy) were added, purging with argon was continued for another 15 min, and then the mixture was heated at T_{b}=120° C. After 16 h the reaction mixture was concentrated to dryness and purified by silica gel column chromatography with 2% ethyl acetate in hexanes as eluent. The purest fractions were combined and concentrated to give of trimethyl-[7-(4,4,5,5-tetramethyl-1,3,2-dioxaborolan-2-yl)pyren-1-yl]silane, Intermediate 5 (16 g, 55% yield), having 99.6% purity by UPLC analysis.

### Synthesis Example 5

This example illustrates the preparation of a compound having Formula 11, Compound 27.

1,3-Dibromo-7-tert-butyl-pyrene (0.606 g, 1.46 mmole), N-phenyl-1-naphthylamine (0.672 g, 3.07 mmole), Pd₂(dba)₃ (0.027 g, 0.029 mmole), tri-tert-butyl-phosphine (0.012 g, 0.058 mmole) and toluene (100 ml) were added to 250 mL round bottom reaction flask at room temperature under nitrogen atmosphere. After that sodium tert-butoxide (0.308 g, 0.321 mmole) was added to the mixture and the resulting suspension stirred at room temperature for 5 min, then heated to 100° C. overnight. The reaction mixture was cooled down to ambient temperature, water (100 ml) added and mixture was stirred in the air for 30 min. After that organic layer was separated and passed through a filter filled with layers of celite, florisil and silica gel washing with toluene (100 mL). Solvent was removed on rotary evaporator, the residue was redissolved in dichloromethane, evaporated onto celite and subjected to separation on silica gel column using mixture of hexanes and dichloromethane as eluent. Chromatography was repeated one time more. All fractions containing the product combined, eluent evaporated, the residue dissolved in toluene and precipitated into methanol. Yield of 7-tert-butyl-N¹,N³-bis(phenyl)-N¹,N³-bis(1-naphthyl)-pyrene-5,9-diamine, Compound 27, 0.162 g (0.23 mmole, 16%). MS: MH+=693. ¹H-NMR: 1.52 (s, 9H), 6.67 (d, 4H, J=8.5 Hz), 6.81 (t, 2H, J=7.5 Hz), 7.01 (br s, 4H), 7.07 (d, 2H, J=8.5 Hz), 7.16-7.27 (m, 7H), 7.3-7.41 (m, 2H), 7.58 (d, 2H, J=2H), 7.77-7.81 (m, 4H), 7.90 (d, 2H, J=7.5 Hz), 8.07 (s, 1H), 8.09 (d, 2H, J=7.5 Hz). Purity by UPLC—>99.9%.

### Synthesis Example 6

This example illustrates the preparation of a compound having Formula II, Compound 28.

Diphenylamine and 1,3-dibromo-7-tert-butylpyrene were transferred into a drybox and placed into 250 ml round bottom flask. After that Pd₂(dba)₃, tri-tert-butyl-phosphine and toluene were added at room temperature followed by sodium tert-butoxide. The resulting suspension was stirred for a short time (ca. 1 min) at ambient temperature, then heated to 80° C. for approx. 2 hours. UPLC analysis of crude reaction mixture after 1.5 hours showed complete conversion of starting bromide into desired product. The mixture was cooled to 60° C. and transferred into fumehood. Water (100 ml) added and the reaction mixture was stirred in the air for 20 min. After that toluene layer separated and passed through a layer of basic alumina, florisil and silica gel washing with toluene (300 mL). Solvent was removed on rotary evaporator and the residue was completely dissolved in ca. 50-100 ml of hexanes. Slowly crystallized after 1 hour product was collected by filtration to afford 1.05 g of crude material with purity 97% by UPLC. This crude product was redissolved in dichloromethane and evaporated onto celite followed by purification on ISCO CombiFlash using hexanes-dichloromethane mixtures as eluent. Fractions (fractions 9-18) containing the product combined, solvents evaporated by using rotary evaporator until volume 10-20 ml. Slowly crystallized product was collected by filtration, dried, redissolved again in ca. 50 ml of toluene and precipitated into ca 300 ml of methanol. Crystals collected by filtration and dried in vacuum to afford 845 mg (1.43 mmol, yield 60%) of the desired product with 99.97% purity by UPLC. MS: MH+=594. ¹H-NMR: 1.53 (s, 9H), 6.62 (d, 4H, J=8.5 Hz), 7.05 (d, 6H, J=8.0 Hz), 7.16-7.19 (m, 9H), 7.25-7.28 (m, 2H), 7.68 (s, 1H), 7.87 (2, 2H, J=9 Hz), 8.07 (d, 2H, J=9 Hz), 8.11 (s, 1H).

### Synthesis Example 7

This example illustrates the preparation of a compound having Formula II, Compound 29.

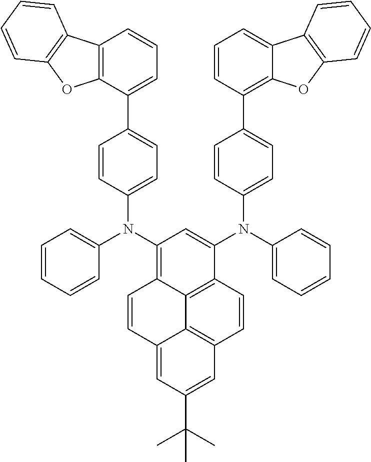 [SMILES: CC(C)(C)c1cc2ccc3c(N(c4ccccc4)c4ccc(-c5cccc6c5oc5ccccc56)cc4)cc(N(c4ccccc4)c4ccc(-c5cccc6c5oc5ccccc56)cc4)c4ccc(c1)c2c34]

4-(Dibenzo[b,d]furan-4-yl)phenyl-N-phenyl amine (1.688 g, 5.046 mmole) and 7-tert-butyl-1,3-dibromo-pyrene (1 g, 2.40 mmole) were transferred into a drybox and placed into 250 ml round bottom flask. After that Pd₂(dba)₃ (44 mg, 0.048 mmole, 2 mol %), tri-tert-butyl-phosphine (19 mg, 0.096 mmole, 4 mol %) and toluene were added to the flask at room temperature followed by sodium tert-butoxide (0.576 g, 6 mmole). The resulting suspension was stirred for a short period (ca. 1 min) at ambient temperature, then heated to 80° C. for approx. 2 hours. UPLC analysis of crude reaction mixture after 1.5 hours showed complete conversion of starting bromide into the desired product. The mixture was cooled to 60° C. and transferred into fumehood. Water (100 ml) added and the reaction mixture was stirred in the air for 20 min. After that toluene layer separated and passed through a layer of basic alumina, florisil and silica gel washing with toluene (300 mL). Solvent was removed on rotary evaporator, the residue was completely dissolved in ca. 30 ml of toluene and precipitated with approx. 150 ml of hexanes. UPLC analysis of precipitated crude product (0.84 g) showed purity of product ca. 99.5%. This crude product was redissolved in dichloromethane and evaporated onto celite followed by purification on ISCO CombiFlash using hexanes-dichloromethane mixtures as eluent. Fractions containing the product combined, solvents evaporated by using rotary evaporator until volume 10-20 ml. Slowly crystallized product was collected by filtration, dried, redissolved again in ca. 50 ml of toluene and precipitated into ca 300 ml of methanol. Yellow crystals collected by filtration and dried in vacuum to afford 370 mg (0.4 mmole, 17%) of the desired product with >99.99% purity by UPLC. MS: MH+=926. ¹H NMR (CDCl₃, 500 MHz): 1.55 (s, 9H), 6.98 (t, 2H, J=7 Hz), 7.15-7.27 (m, 14H), 7.31-7.35 (m, 4H), 7.39 7.43 (m, 2H), 7.53-7.55 (m, 4H), 7.79 (d, 4H, J=8.5 Hz), 7.84-7.86 (m, 3H), 7.94-7.96 (m, 3H), 8.15-8.18 (m, 3H).

### Synthesis Example 8

This example illustrates the preparation of a compound having Formula II, Compound 30.

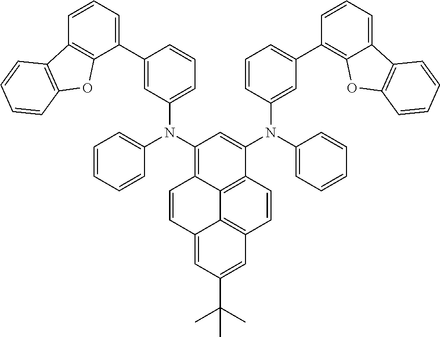 [SMILES: CC(C)(C)c1cc2ccc3c(N(c4ccccc4)c4cccc(-c5cccc6c5oc5ccccc56)c4)cc(N(c4ccccc4)c4cccc(-c5cccc6c5oc5ccccc56)c4)c4ccc(c1)c2c34]

3-(Dibenzo[b,d]furan-4-yl)phenyl-N-phenyl-amine (1.61 g, 4.805 mmole) and 1,3-dibromo-7-tert-butylpyrene (0.95 g, 2.28 mmole) were placed into 250 ml round bottom flask. After that Pd₂(dba)₃ (44 mg, 0.048 mmole), tri-tert-butyl-phosphine (19 mg, 0.096 mmole) and toluene were added at room temperature followed by sodium tert-butoxide (0.576 g, 6 mmole). The resulting suspension was stirred for a short period (ca. 1 min) at ambient temperature, then heated to 80° C. for approx. 4 hours. UPLC analysis of crude reaction mixture after 2 hours showed complete conversion of starting bromide into desired product. The mixture was cooled to 60° C. and transferred into fumehood. Water (100 ml) added and the reaction mixture was stirred in the air for 20 min. After that toluene layer separated and passed through layers of celite, basic alumina, florisil and silica gel washing with toluene (300 mL). Solvent was removed on rotary evaporator and the residue was redissolved in dichloromethane and evaporated onto celite followed by purification on ISCO column chromatography using hexanes—dichloromethane mixture as an eluent. Fractions containing material combined together, solvent evaporated using rotary evaporator until residual volume ca. 10 ml. The product collected by filtration, redissolved in 30 ml of toluene and precipitated in ca. 300 ml of methanol to yield totally 1.04 g (1.12 mmole, 49%) of the product as yellowish crystalline solids with purity greater than 99.5% by UPLC. MS: MH+=926. ¹H NMR (CDCl₃, 500 MHz): 1.53 (s, 9H), 6.69 (t, 2H, J=7 Hz), 7.10-7.19 (m, 12H), 7.24-7.34 (m, 8 H), 7.38-7.43 (m, 4H), 7.66 (br. s, 2H), 7.82 (dd, 2H, J1=1 Hz, J2=7.5 Hz), 7.88-7.94 (m, 5H), 8.14 (s, 2H), 8.20 (d, 2H, J=9 Hz).

### Synthesis Example 9

This example illustrates the preparation of a compound having Formula I, Compound 15.

a. Synthesis of N-(2,4-dimethylphenyl)-9-phenyl-9H-carbazol-2-amine (Intermediate 6).

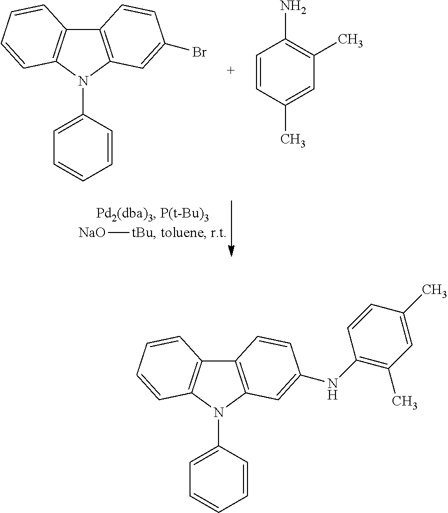

To 5.0 g of 2-bromo-9-phenylcarbazole, 1.97 g of 2,4-dimethylaniline, 284 mg of Pd₂(dba)₃, and 125 mg of P(t-Bu)₃ in 90 mL toluene was added 1.57 g of sodium tert-butoxide. The reaction was stirred at room temperature. After 19 hours, water was added and the contents were extracted with dichloromethane. The combined extracts were dried over sodium sulfate, and concentrated by rotary evaporation to give a solid. The crude material was dissolved in dichloromethane and purified by MPLC on silica gel, eluting with 90:10 to 70:30 hexanes:dichloromethane, combining the purest fractions to give, after concentration by rotary evaporation, Intermediate 6 (3.2 g, 57% yield) as a near-colorless liquid.

b. Synthesis of 1-Trimethylsilyl-7-bromopyrene (Intermediate 7).

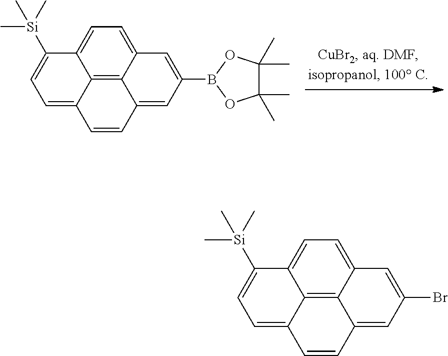

A mixture of 4.0 g of Intermediate 5 (from Synthesis Example 4) in 120 mL isopropanol and 20 mL DMF was treated with 3.35 g of CuBr₂ in 20 mL of water. The reaction was heated at T_{b}=100° C. under nitrogen. After 23 hours, an additional 4.1 g of CuBr₂ and 20 mL DMF were added. After 7 h the reaction was complete. The reaction was cooled to room temperature, 250 mL of water was added, and the mixture filtered. The solid was washed with 100 mL water, then 50 mL methanol, to give a beige solid. Chloroform was added to dissolve the solid and the filtrate was concentrated by rotary evaporation. The solid was dissolved in 20 mL chloroform and passed through plug of a silica gel eluting with 3:1 hexanes:dichloromethane to give Intermediate 7 solid (3.4 g, 59% yield) as a white solid.

c. Synthesis of 1-Trimethylsilyl-7-(2,2′,4,4′-tetramethyldiphenylamino)pyrene (Intermediate 8)

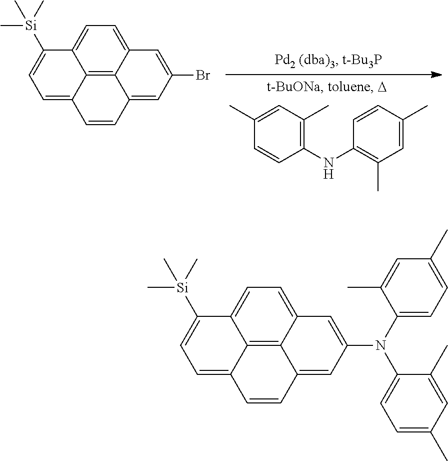

To 5.8 g of Intermediate 7 in 80 mL toluene under nitrogen atmosphere was added 194 mg of P(t-Bu)₃ and 440 mg of Pd₂(dba)₃. Next, 4.73 g of Intermediate 3 and 2.02 g of sodium tert-butoxide were added. The reaction was heated at T_{b}=104° C. for 4 hours. After the reaction was cooled, water was added and the contents were extracted with dichloromethane. The combined extracts were dried over sodium sulfate, filtered, and concentrated by rotary evaporation. The crude material was dissolved in dichloromethane and purified by MPLC eluting with 95:5 to 70:30 hexanes:dichloromethane. The less pure fractions were re-purified by MPLC on silica gel, eluting with 95:5 to 80:20 hexanes:dichloromethane. The combined lots provided Intermediate 8 (7.3 g, 89% yield) as a yellow solid.

d. Synthesis of 1-Iodo-7-(2,2′,4,4′-tetramethyldiphenylamino)pyrene (Intermediate 9)

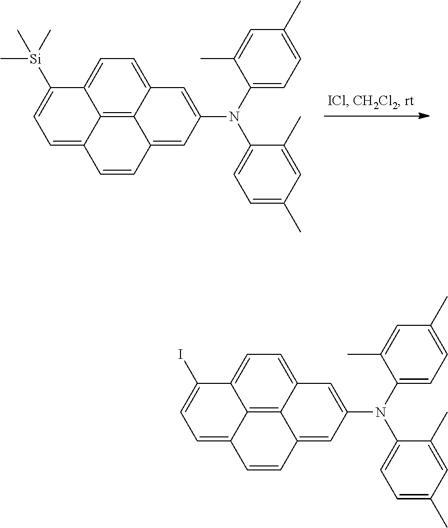

To 5.0 g of Intermediate 8 suspended in 140 mL DCM was added 1.7 g of ICI in 10.0 mL DCM. The reaction was stirred at room temperature. After 2 hours, water and sat. aq. sodium sulfite were added, and the contents were extracted with dichloromethane. The combined extracts were dried over sodium sulfate, filtered, and concentrated by rotary evaporation. The crude material was dissolved in dichloromethane and purified by on silica gel, eluting with 95:5 hexanes:dichloromethane to give Intermediate 9 (3.23 g, 62% yield) as a yellow foam.

e. Compound 15.

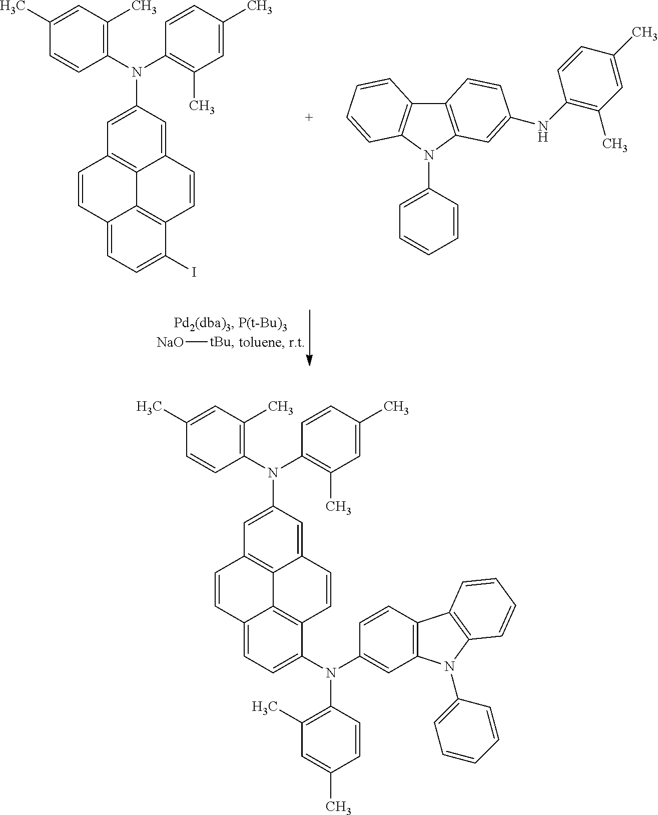

To a mixture of 3.2 g of Intermediate 9 in 14 mL toluene under nitrogen atmosphere was added 70 mg of P(t-Bu)₃ and 158 mg of Pd₂(dba)₃. Next, 2.5 mg of Intermediate 6 and 672 mg of sodium tert-butoxide were added. The reaction was heated at T_{b}=71° C. for 90 minutes. After the reaction was cooled, water was added and the contents were extracted with dichloromethane. The combined extracts were dried over sodium sulfate, filtered, and concentrated by rotary evaporation to give a brown foam. The crude material was dissolved in dichloromethane and purified by MPLC on silica gel, eluting with 93:7 to 65:35 hexanes:dichloromethane. The purest fractions were combined and concentrated by rotary evaporation to give the product (1.85 g, 99.33% pure). The material was dissolved in toluene and passed through a plug of basic alumina/Florisil eluting with toluene to give the product at a higher purity (99.55% pure). The material was dissolved in toluene and passed through a plug of basic alumina/Florisil eluting with 1:1 hexanes:toluene to give Compound 15 (920 mg, 20% yield, 99.80% pure) as yellow solid. Final purification prior to device preparation was accomplished by vacuum sublimation.

### Synthesis Example 10

This example illustrates the preparation of a compound having Formula II, Compound 47.

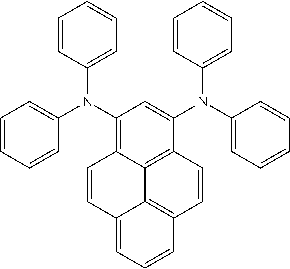 [SMILES: c1ccc(N(c2ccccc2)c2cc(N(c3ccccc3)c3ccccc3)c3ccc4cccc5ccc2c3c54)cc1]

Compound 28 from Synthesis Example 6 (0.3 g, 0.506 mmole) was dissolved in in approx. 100 ml of benzene. After that 0.067 g of AlCl₃ was added at once resulting in emerald-green solution. Reaction mixture stirred at ambient temperature for overnight. Additional portion of AlCl₃ (0.35 g) added at once and the resulting mixture stirred at ambient temperature for additional 2 days. Reaction was quenched with acetone, water, organic phase separated and passed through filter filled with silica gel. The residue after evaporation of solvent was subjected to ISCO chromatography on silica gel using hexanes dichloromethane mixtures as eluent. Fractions containing pyrenyl compound combined, eluent evaporated to volume approx. 10-20 ml, precipitate collected by filtration to give approx. 60 mg of Compound 47 with purity 98.5% by UPLC. MS: MH+=537. ¹H NMR (CDCl₃, 500 MHz): 6.92 (br. m, 4H), 7.05 (d, 8H, J=8 Hz), 7.17 (t, 8H, J=8 Hz), 7.73 (s, 1H), 7.9-8.08 (br. m, 5H), 8.12 (d, 2H, J=9 Hz).

### Device Examples

### (1) Materials

D-1 is a blue benzofluorene dopant. Such materials have been described, for example, in U.S. Pat. No. 8,465,848.
D-2 is a blue benzofluorene dopant. Such materials have been described, for example, in U.S. Pat. No. 8,465,848.
ET-1 is an aryl phosphine oxide.
ET-2 is lithium quinolate.
HIJ-1 is a hole injection material which is made from an aqueous dispersion of an electrically conductive polymer and a polymeric fluorinated sulfonic acid. Such materials have been described in, for example, U.S. Pat. No. 7,351,358.
Host H1 is a deuterated anthracene compound.
Host H2 is an aryl-anthracene compound.
Host H3 is a heteroaryl-anthracene compound.
Host H4 is a deuterated anthracene compound.
HTM-1 is a hole transport material which is a triarylamine polymer. Such materials have been described in, for example, published US Application 2013-0082251.

### (2) Device Fabrication

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen.

Immediately before device fabrication the cleaned, patterned ITO substrates were treated with UV ozone for 10 minutes. Immediately after cooling, an aqueous dispersion of HIJ-1 was spin-coated over the ITO surface and heated to remove solvent. After cooling, the substrates were then spin-coated with a solvent solution of HT-1, and then heated to remove solvent.

In some examples, after formation of the hole transport layer, the workpieces were then spin-coated with a solution of the photoactive layer materials in methyl benzoate and heated to remove solvent. The workpieces were then masked and place in a vacuum chamber. A layer of ET-1 was deposited by thermal evaporation, followed by a layer of EIJ-1. Masks were then changed in vacuo and a layer of Al was deposited by thermal evaporation. The chamber was vented, and the devices were encapsulated using a glass lid, desiccant, and UV curable epoxy. In some examples, after formation of the hole transport layer, the workpieces were masked and placed in a vacuum chamber. The materials in the photoactive layer were then deposited by thermal evaporation. A layer of ET-1 was then deposited by thermal evaporation, followed by a layer of EIJ-1. Masks were then changed in vacuo and a layer of Al was deposited by thermal evaporation. The chamber was vented, and the devices were encapsulated using a glass lid, desiccant, and UV curable epoxy.

### (3) Device Characterization

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The power efficiency is the current efficiency divided by the operating voltage. The unit is lm/W. The color coordinates were determined using either a Minolta CS-100 meter or a Photoresearch PR-705 meter.

### Device Examples 1-3 and Comparative Example A

These examples illustrate the use of a material having Formula I as the photoactive dopant in a device where the photoactive layer is applied by solution deposition.

The host was H1.

In Examples 1-3, the dopant was Compound 2.

In Comparative Example A, the dopant was D-1.

Device results are given in Table 1.

Device structure, in order (all percentages are by weight, based on the total weight of the layer):
Glass substrate
Anode: ITO (50 nm)
Hole injection layer: HIJ-1 (42 nm)
Hole transport layer: HTM-1 (18 nm)
Photoactive layer: Host and dopant in the ratio given in Table 1 (38 nm)
Electron transport layer: ET-1 (8 nm)
Electron injection layer: ET-2 (12 nm)
Cathode: Al (100 nm)

TABLE 1
Device results

| | | | CE | EQE | | |
|---|---|---|---|---|---|---|
| Ex. | Dopant | Ratio | Cd/A | (%) | V | CIE (x, y) |
| 1 | Comp. 2 | 93:7 | 2.6 | 3.2 | 4.2 | 0.144, 0.090 |
| 2 | Comp. 2 | 98.8:1.2 | 1.9 | 2.5 | 4.2 | 0.148, 0.084 |
| 3 | Comp. 2 | 96.5:3.5 | 2.4 | 3.1 | 4.2 | 0.145, 0.085 |
| A | D-1 | 93:7 | 6.3 | 5.8 | 4.6 | 0.140, 0.130 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 1 illustrates the use of Compound 2 as a dopant in the emissive layers of organic electronic devices with deep blue color.

### Device Example 4 and Comparative Example B

This example illustrates the use of a material having Formula I as the photoactive dopant in a device where the photoactive layer is vapor deposited.

Except for the photoactive layer, the device layers were as described above for Examples 1-3.

In Example 4, the photoactive layer was 20 nm of vapor deposited host H1 and Compound 2, in a 20:1 weight ratio.

In Comparative Example B, the photoactive layer was 20 nm of vapor deposited host H1 and dopant D-2, in a 13:1 weight ratio.

The device results are given in Table 2.

TABLE 2
Device results

| | | | CE | EQE | | |
|---|---|---|---|---|---|---|
| Ex. | Dopant | Ratio | Cd/A | (%) | V | CIE (x, y) |
| 4 | Comp. 2 | 20:1 | 2.8 | 3.8 | 4.0 | 0.144, 0.077 |
| B | D-2 | 13:1 | 8.2 | 8.0 | 4.0 | 0.140, 0.119 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 2 illustrates the use of Compound 2 as a dopant in the vapor-deposited emissive layer of organic electronic devices with deep blue color.

### Device Examples 5-6

These examples illustrate the use of a compound having Formula I as the photoactive dopant with different hosts, where the photoactive layer is applied by solution deposition.

Except for the photoactive layer, the device layers were as described above for Examples 1-3.

In Example 5, the photoactive layer was host H2 and Compound 2 in a 93:7 weight ratio (38 nm).

In Example 6, the photoactive layer was host H3 and Compound 2 in a 96:4 weight ratio (38 nm).

The device results are given in Table 3.

TABLE 3
Device results

| | | | CE | EQE | | |
|---|---|---|---|---|---|---|
| Ex. | Dopant | Host | Cd/A | (%) | V | CIE (x, y) |
| 5 | Comp. 2 | H2 | 2.9 | 3.8 | 4.8 | 0.143, 0.080 |
| 6 | Comp. 2 | H3 | 2.9 | 3.7 | 4.5 | 0.143, 0.084 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 3 illustrates the use of Compound 2 as a dopant in the emissive layers of organic electronic devices with deep blue color.

### Device Examples 7-9 and Comparative Example C

These examples illustrate the use of a compound having Formula I, Compound 5, as the photoactive dopant, where the photoactive layer is applied by solution deposition.

Except for the photoactive layer, the device layers were as described above for Examples 1-3.

The host was H1. The dopants and the ratios are given in Table 4.

The device results are given in Table 4.

TABLE 4
Device results

| | | | CE | EQE | | |
|---|---|---|---|---|---|---|
| Ex. | Dopant | Ratio | Cd/A | (%) | V | CIE (x, y) |
| 7 | Comp. 5 | 93:7 | 5.0 | 5.3 | 4.6 | 0.139, 0.108 |
| 8 | Comp. 5 | 98:2 | 4.3 | 4.9 | 4.6 | 0.148, 0.084 |
| 9 | Comp. 5 | 96.5:3.5 | 4.9 | 5.4 | 4.6 | 0.140, 0.101 |
| C | D-1 | 93:7 | 5.8 | 5.5 | 4.6 | 0.140, 0.126 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 4 illustrates the use of Compound 5 as a dopant in the emissive layers of organic electronic devices with deep blue color.

### Device Examples 10-12 and Comparative Example D

These examples illustrate the use of a compound having Formula II, Compound 27, as the photoactive dopant, where the photoactive layer is applied by solution deposition.

Except for the photoactive layer, the device layers were as described above for Examples 1-3.

The host was H1. The dopants and the ratios are given in Table 5.

The device results are given in Table 5.

TABLE 5
Device results

| | | | CE | EQE | | |
|---|---|---|---|---|---|---|
| Ex. | Dopant | Ratio | Cd/A | (%) | V | CIE (x, y) |
| 10 | Comp. 27 | 93:7 | 4.5 | 4.9 | 4.7 | 0.138, 0.107 |
| 11 | Comp. 27 | 96:4 | 4.4 | 4.9 | 4.7 | 0.139, 0.103 |
| 12 | Comp. 27 | 98:2 | 3.9 | 4.5 | 4.5 | 0.140, 0.097 |
| D | D-1 | 93:7 | 6.4 | 5.7 | 4.7 | 0.139, 0.139 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 5 illustrates the use of Compound 27 as a dopant in the emissive layers of organic electronic devices with deep blue color.

### Device Example 13 and Comparative Example E

This example illustrates the use of a compound having Formula II as the photoactive dopant, where the photoactive layer is vapor deposited.

Except for the photoactive layer, the device layers were as described above for Examples 1-3.

In Example 13, the photoactive layer was 20 nm of vapor deposited host H1 and Compound 27, in a 13:1 weight ratio.

In Comparative Example E, the photoactive layer was 20 nm of vapor deposited host H1 and dopant D-2, in a 13:1 weight ratio.

The device results are given in Table 6.

TABLE 6
Device results

| | | | CE | EQE | | |
|---|---|---|---|---|---|---|
| Ex. | Dopant | Ratio | Cd/A | (%) | V | CIE (x, y) |
| 13 | Comp. 27 | 13:1 | 5.2 | 5.5 | 4.4 | 0.137, 0.107 |
| E | D-2 | 13:1 | 8.9 | 8.2 | 4.4 | 0.138, 0.131 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 6 illustrates the use of Compound 27 as a dopant in the vapor-deposited emissive layer of an organic electronic device with deep blue color.

### Device Example 14

This example illustrates the use of a compound having Formula II as the photoactive dopant with a different host, where the photoactive layer is applied by solution deposition.

Except for the photoactive layer, the device layers were as described above for Examples 1-3.

In Example 14, the photoactive layer was host H4 and Compound 27, in a 96:4 weight ratio (38 nm).

The device results are given in Table 7.

TABLE 7
Device results

| | | | CE | EQE | | |
|---|---|---|---|---|---|---|
| Ex. | Dopant | Host | Cd/A | (%) | V | CIE (x, y) |
| 14 | Comp. 27 | H4 | 4.1 | 5.0 | 5.1 | 0.141, 0.087 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 7 illustrates the use of Compound 27 as a dopant in the emissive layer of an organic electronic device with deep blue color.

### Device Examples 15 and 16 and Comparative Example F

This example illustrates the use of a compound having Formula II as the photoactive dopant with a different host, where the photoactive layer is applied by solution deposition.

Except for the photoactive layer, the device layers were as described above for Examples 1-3.

The host was H1. The dopants and the ratios are given in Table 8.

The device results are given in Table 8.

TABLE 8
Device results

| | | | CE | EQE | | |
|---|---|---|---|---|---|---|
| Ex. | Dopant | Ratio | Cd/A | (%) | V | CIE (x, y) |
| 15 | Comp. 28 | 93:7 | 4.8 | 5.4 | 4.5 | 0.140, 0.098 |
| 16 | Comp. 28 | 96:4 | 4.6 | 5.3 | 4.5 | 0.140, 0.096 |
| F | D-1 | 93:7 | 6.3 | 5.8 | 4.7 | 0.139, 0.139 |

All data at 1000 nit. Ratio is the weight ratio of host to dopant; CE is the current efficiency; EQE=external quantum efficiency; V is the voltage @ 15 mA/cm²; CIE(x,y) refers to the x and y color coordinates according to the C.I.E. chromaticity scale (Commission Internationale de L'Eclairage, 1931).

Table 8 illustrates the use of Compound 28 as a dopant in the emissive layer of an organic electronic device with deep blue color.

### PL Examples 1-3 and Comparatives G and H

These examples illustrate the photoluminescence of compounds having Formula I.

The compounds were individually dissolved in toluene. The concentration was adjusted such that the optical density of the solution in a 1-cm quartz cell was preferably in the 0.2-0.4 range, at the excitation wavelengths between 300 and 360 nm. The photoluminescence spectrum was measured with a Spex Fluorolog spectrometer. The results are given in Table 9 below, where “PL” indicates photoluminescence.

TABLE 9

| | | Concentration, | PL peak, | PL FWHM, |
|---|---|---|---|---|
| Example | Compound | μM | nm | nm |
| PL1 | 2 | 7.5 | 445 | 47 |
| PL2 | 5 | 15 | 450 | 50 |
| PL3 | 24 | 5 | 456 | 50 |
| G | D-1 | 5 | 454 | 57 |
| H | D-2 | 13 | 449 | 57 |

It can be seen from the data in Table 9, that the compounds of Formula I have a narrower and more desirable FWHM as compared to the benzofluorene compounds.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A compound having Formula III 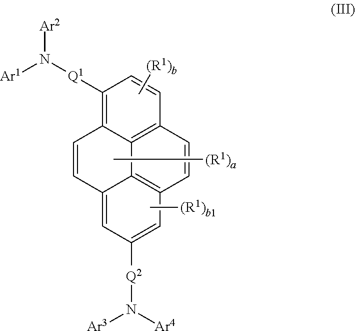
wherein:
Q¹ and Q² are the same or different and are selected from a single bond, hydrocarbon aryl and deuterated hydrocarbon aryl;
Ar¹-Ar⁴ are the same or different and are selected from the group consisting of hydrocarbon aryl, heteroaryl, substituted derivatives thereof, and deuterated analogs thereof, wherein Ar¹ and Ar² may be joined to form a carbazole group and Ar³ and Ar⁴ may be joined to form a carbazole group;
R¹ is the same or different at each occurrence and is selected from the group consisting of D, F, CN, alkyl, alkoxy, fluoroalkyl, hydrocarbon aryl, aryloxy, heteroaryl, silyl, siloxane, siloxy, germyl, deuterated alkyl, deuterated partially-fluorinated alkyl, deuterated alkoxy, deuterated hydrocarbon aryl, deuterated aryloxy, deuterated heteroaryl, deuterated heteroaryl deuterated silyl, deuterated siloxane, deuterated siloxy, and deuterated germyl;
a is an integer of 0-4; and
b and b1 are the same or different and are an integer of 0-2.

2. The compound of claim 1, wherein Q¹ and Q² are a single bond.

3. The compound of claim 1, wherein Ar¹ is selected from the group consisting of phenyl, biphenyl, terphenyl, napthyl, naphthylphenyl, phenylnaphthyl, styryl, derivatives thereof having one or more substituents selected from the group consisting of fluoro, alkyl, alkoxy, silyl, siloxy, and deuterated analogs thereof.

4. The compound of claim 1, wherein Ar¹ is a hydrocarbon aryl and has at least one substituent that is an N-heteroaryl or deuterated N-heteroaryl having at least one ring atom which is N.

5. The compound of claim 1, wherein Ar¹ is a hydrocarbon aryl and has at least one substituent that is an O-heteroaryl or deuterated O-heteroaryl having at least one ring atom which is O.

6. The compound of claim 1, wherein Ar¹ is an N-heteroaryl or deuterated N-heteroaryl having at least one ring atom which is N.

7. The compound of claim 1, wherein Ar¹ is an O-heteroaryl or deuterated O-heteroaryl having at least one ring atom which is O.

8. The compound of claim 1, wherein Ar¹═Ar³ and Ar²═Ar⁴.

9. The compound of claim 2, wherein the compound has differently-substituted amino groups.

10. The compound of claim 1, wherein at least one of Q¹ and Q² is a hydrocarbon aryl or substituted hydrocarbon aryl group and at least one of Q¹ and Q² is a single bond.

11. The compound of claim 1, wherein Q¹ and Q² are hydrocarbon aryl or deuterated hydrocarbon aryl, and further wherein the compound has differently-substituted aryl-amino groups.

12. The compound of claim 1, wherein the compound has Formula III-a or Formula III-b

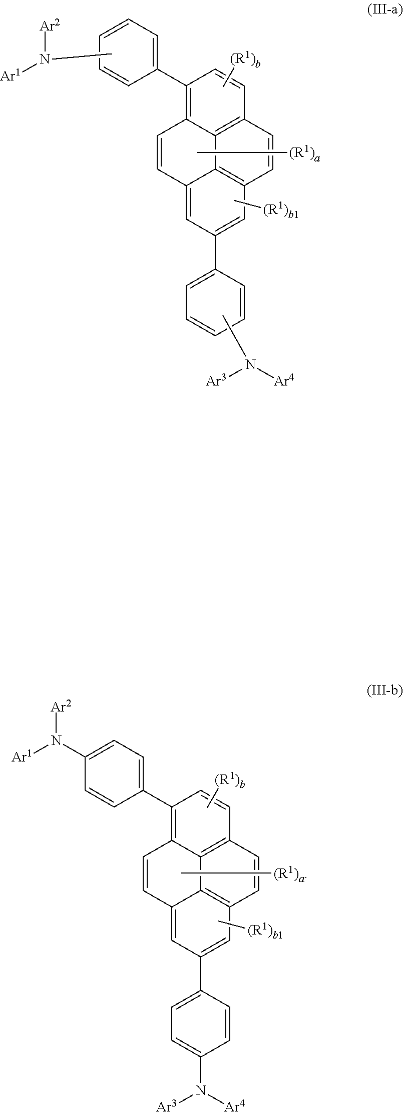

13. An electronic device comprising at least one photoactive layer, wherein the photoactive layer comprises the compound of claim 1.
